(19) United States
(12) Patent Application Publication
(10) Pub. No.: US 2025/0163009 A1
(43) Pub. Date: May 22, 2025

(54) BENZOTHIA(DIA)ZEPINE COMPOUNDS FOR TREATMENT OF HBV AND HDV

(71) Applicant: Assembly Biosciences, Inc., South San Francisco, CA (US)

(72) Inventors: Jiaxin YU, South San Francisco, CA (US); Min ZHONG, South San Francisco, CA (US); Michael WALKER, South San Francisco, CA (US); Mark BURES, South San Francisco, CA (US); Hassan PAJOUHESH, South San Francisco, CA (US); Ken ZHANG, South San Francisco, CA (US)

(21) Appl. No.: 18/841,305

(22) PCT Filed: Feb. 24, 2023
(86) PCT No.: PCT/US2023/013891

§ 371 Date: Aug. 23, 2024

Related U.S. Application Data
(60) Provisional application No. 63/314,109, filed on Feb. 25, 2022.

(51) Int. Cl. C07D 285/36; A61K 31/554; C07D 417/04; C07D 417/10

(52) U.S. Cl. CPC C07D 285/36; A61K 31/554; C07D 417/04; C07D 417/10

(57) ABSTRACT

The present disclosure provides, in part, benzothia(dia)zepine compounds, and pharmaceutical compositions thereof, and methods of treating Hepatitis B (HBV) and Hepatitis D (HDV) infections.

### CROSS-REFERENCE TO RELATED APPLICATION

This application claims the benefit of U.S. Provisional Application No. 63/314,109, filed Feb. 25, 2022, the contents of which are hereby incorporated by reference.

### BACKGROUND

Hepatitis B (HBV) causes viral hepatitis that can further lead to chronic liver disease and increase the risk of liver cirrhosis and liver cancer (hepatocellular carcinoma). Worldwide, about 2 billion people have been infected with HBV, around 360 million people are chronically infected, and every year HBV infection causes more than one half million deaths. HBV can be spread by body fluids: from mother to child, by sex, and via blood products. Children born to HBV-positive mothers may also be infected, unless vaccinated at birth.

The hepatitis virus particle is composed of a lipid envelope studded with surface protein (HBsAg) that surrounds the viral core. The core is composed of a protein shell, or capsid, built of 120 core protein (Cp) dimers, which in turn contains the relaxed circular DNA (rcDNA) viral genome as well as viral and host proteins. In an infected cell, the genome is found as a covalently closed circular DNA (cccDNA) in the host cell nucleus. The cccDNA is the template for viral RNAs and thus viral proteins. In the cytoplasm, Cp assembles around a complex of full-length viral RNA (the so-called pregenomic RNA or pgRNA and viral polymerase (P). After assembly, P reverse transcribes the pgRNA to rcDNA within the confines of the capsid to generate the DNA-filled viral core.

At present, chronic HBV is primarily treated with nucleos(t)ide analogs (e.g., entecavir) that suppress the virus while the patient remains on treatment, but do not eliminate the infection, even after many years of treatment. Once a patient starts taking nucleos(t)ide analogs, most must continue taking them or risk the possibility of a life-threatening immune response due to viral rebound. Further, nucleotide therapy may lead to the emergence of antiviral drug resistance.

The only FDA approved alternative to nucleos(t)ide analogs is treatment with interferon α or pegylated interferon α. Unfortunately, the adverse event incidence and profile of interferon α can result in poor tolerability, and many patients are unable to complete therapy. Moreover, only a small percentage of patients are considered appropriate for interferon therapy, as only a small subset of patients is likely to have a sustained clinical response to a course of interferon therapy. As a result, interferon-based therapies are used in only a small percentage of all diagnosed patients who elect treatment.

Thus, current HBV treatments can range from palliative to watchful waiting. Nucleotide analogs suppress virus production, treating the symptom, but leave the infection intact. Interferon α has severe side effects and less tolerability among patients and is successful as a finite treatment strategy in only a small minority of patients. There is a clear on-going need for more effective treatments for HBV infections.

Another form of viral hepatitis is Hepatitis D virus (HDV), a defective RNA virus that causes chronic viral hepatitis and eventual cirrhosis. However, the HDV life cycle is dependent on the presence of HBsAg for assembly. Thus, in a small set of patients infected with HBV, HDV presents as coinfection with HBV. See for example, Sagnelli et al., Life (Basel). 2021 February; 11(2): 169, Published online 2021 Feb. 22. doi: 10.3390/life11020169, herein incorporated by reference with regard to such background teaching. For patients already infected with HBV, coinfection with HDV can further exacerbate the symptoms of HBV, increasing the likelihood of complications, rapid disease progression and/or death. Chronic HBV/HDV infection is also associated with the development of hepatocellular carcinoma (HCC). Like HBV, treatment options for HDV infection or HBV/HDV coinfection, are limited and include those used to treat HBV. Thus, there is a need for effective therapeutic options for the treatment of HDV infection or HBV/HDV coinfection.

WO2021/110883, WO2021/110884, WO2021/110885, WO2021/110886, WO2021/110887, WO2020/161216, WO2020/161217 and WO2019/234077 relate to 1,5-benzothiazepine and 1,2,5-benzothiadiazepine derivatives described as bile acid modulators having apical sodium-dependent bile acid transporter (ASBT) and/or liver bile acid transport (LBAT) inhibitory activity. The compounds are further described as being useful to in the treatment of cardiovascular diseases, fatty acid metabolism and glucose utilization disorders, gastrointestinal diseases and liver diseases.

LBAT functions as a cellular receptor for viral entry of the hepatitis B virus (HBV) and hepatitis D virus (HDV), which in turn is the major cause of liver disease and hepatocellular carcinoma. There is a need for additional bile acid modulating compounds that have an improved profile with respect to potency, selectivity and/or bioavailability.

### SUMMARY

The present disclosure provides, in part, benzothia(dia)zepine compounds and pharmaceutical compositions thereof, useful for inhibition of HBV or HDV replication, inhibition of HBV or HDV viral entry, and methods of treating HBV infections, HDV infection or HBV/HDV coinfection.

In one aspect, the disclosure provides a compound of Formula I:

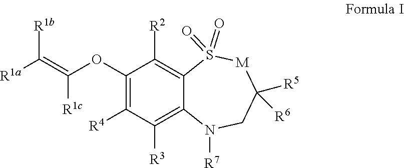

or a pharmaceutically acceptable salt thereof, where the variables are described in the detailed description.

In another aspect, the disclosure provides pharmaceutical compositions comprising a compound of Formula I, or a pharmaceutically acceptable salt thereof, and a pharmaceutically acceptable excipient.

In another aspect, the disclosure provides a method of treating an HBV infection in a subject in need thereof, comprising: administering to the subject a therapeutically effective amount of compound of Formula I, or a pharmaceutically acceptable salt thereof.

In another aspect, the disclosure provides a method of treating an HBV infection in a subject in need thereof, comprising: administering to the subject a pharmaceutical composition comprising a therapeutically effective amount of a compound of Formula I, or a pharmaceutically acceptable salt thereof, and a pharmaceutically acceptable excipient.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### I. Definitions

The term “alkenyl” as used herein refers to an unsaturated straight or branched hydrocarbon having at least one carbon-carbon double bond. Exemplary alkenyl groups include, but are not limited to, a straight or branched group of 2-6 carbon atoms, referred to herein as C₂₋₆alkenyl. Exemplary alkenyl groups include, but are not limited to, vinyl, allyl, butenyl, and pentenyl, etc.

The term “alkoxy” as used herein refers to a straight or branched alkyl group attached to oxygen (i.e., alkyl-O—). Exemplary alkoxy groups include, but are not limited to, alkoxy groups of 1-6 or 1-4 carbon atoms, referred to herein as C₁₋₆alkoxy and C₁₋₄alkoxy, respectively. Exemplary alkoxy groups include, but are not limited to methoxy, ethoxy, and isopropoxy, etc.

The term “alkoxyalkyl” as used herein refers to an alkyl group substituted with an alkoxy group. Examples include, but are not limited to, CH₃CH₂OCH₂—, CH₃OCH₂CH₂— and CH₃OCH₂—, etc.

The term “alkyl” as used herein refers to a saturated straight or branched hydrocarbon. Exemplary alkyl groups include, but are not limited to, straight or branched hydrocarbons of 1-6 or 1-4 carbon atoms, referred to herein as C₁₋₆ alkyl and C₁₋₄ alkyl, respectively. Exemplary alkyl groups include, but are not limited to, methyl, ethyl, n-propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, t-butyl, n-pentyl, isopentyl, neopentyl, and n-hexyl, etc. The term “alkylene” as used herein refers to a biradical alkyl group.

The term “alkynyl” as used herein refers to an unsaturated straight or branched hydrocarbon having at least one carbon-carbon triple bond. Exemplary alkynyl groups include, but are not limited to, straight or branched groups of 2-6 carbon atoms, referred to herein as C₂₋₆alkynyl. Exemplary alkynyl groups include, but are not limited to, ethynyl, propynyl, butynyl, pentynyl, hexynyl, and methylpropynyl, etc.

The term “carbonyl” as used herein refers to the biradical —C(O)—.

The term “coinfection” as used herein refers to simultaneous infection of a host by more than one viral pathogen.

The term “cyano” as used herein refers to the radical —CN.

The term “cycloalkyl” as used herein refers to a saturated monocyclic hydrocarbon group of, for example, 3-7 carbons, referred to herein as C₃₋₇monocycloalkyl, or bicyclic hydrocarbon ring structure of, for example, 5-12 carbons, referred to herein as C₅₋₁₂bicycloalkyl. For bicyclic cycloalkyl groups, the two rings may be attached through the same or different carbons. Exemplary monocycloalkyl groups include, but are not limited to, cycloheptyl, cyclohexyl, cyclopentyl, cyclopentenyl, cyclobutyl and cyclopropyl. Exemplary bicycloalkyl groups include, but are not limited to, spiro[2.5]octanyl, spiro[3.5]nonanyl, spiro[4.5]decanyl, spiro[5.5]undecanyl, spiro[2.4]heptanyl, spiro[3.4]octanyl, spiro[4.4]nonanyl, spiro[2.3]hexanyl, spiro[3.3]heptanyl, decahydronaphthalene, octahydro-1H-indene, bicyclo[4.2.0]octanyl, bicyclo[4.1.0]heptanyl, octahydropentalenyl, bicyclo[3.2.0]heptanyl, bicyclo[3.1.0]hexanyl, bicyclo[2.2.2]octanyl, bicyclo[2.2.1]heptanyl, bicyclo[3.1.1]heptanyl, and bicyclo[1.1.1]pentanyl.

The terms “halo” or “halogen” as used herein refer to F, Cl, Br or I.

The term “haloalkyl” as used herein refers to an alkyl group substituted with one or more halogen atoms. For example, haloC₁₋₆alkyl refers to a straight or branched alkyl group of 1-6 carbon atoms substituted with one or more halogen atoms. Examples include, but are not limited to, —CH₂F, —CHCl₂, —CHF₂, —CF₃, CF₃CH₂—, CH₃CF₂, CF₃CCl₂— and CF₃CF₂—.

The term “haloalkoxy” as used herein refers to an alkoxy group substituted with one or more halogen atoms. Examples include, but are not limited to, CCl₃O—, CF₃O—, CHF₂O—CF₃CH₂O—, and CF₃CF₂O—.

The terms “heteroaryl” as used herein refers to a 5-6 membered monocyclic aromatic group, referred to herein as monocyclo₅₋₆heteroaryl, or 8-12 membered bicyclic aromatic ring system, referred to herein as bicyclo₈₋₁₂heteroaryl, containing one to four independently selected heteroatoms, such as nitrogen, oxygen and sulfur. Where possible, the heteroaryl ring may be linked to the adjacent radical though carbon or nitrogen. Examples of monocyclo₅₋₆heteroaryl groups include, but are not limited to, furanyl, thiophenyl (also referred to as thienyl), pyrrolyl, thiazolyl, oxazolyl, isothiazolyl, isoxazolyl, imidazolyl, pyrazolyl, 1H-1,2,3-triazolyl, 2H-1,2,3-triazolyl, 1,2,4-triazolyl, pyridinyl (also referred to as pyridyl), pyridazinyl, pyrimidinyl, pyrazinyl, 1,3,5-triazinyl, 1,2,4-triazinyl, 1,2,3-triazinyl, 1,2,4-oxadiazolyl, 1,3,4-oxadiazolyl, 1,2,5-oxadiazolyl, 1,2,4-thiadiazolyl, 1,3,4-thiadiazolyl, 1,2,5-thiadiazolyl and tetrazolyl. Examples of bicyclo₈₋₁₂heteroaryl groups include, but are not limited to, benzofuranyl, isobenzofuranyl, benzo[b]thiophenyl, benzo[c]thiophenyl, indolyl, isoindolyl, benzo[d]isoxazolyl, benzo[c]isoxazolyl, benzo[d]oxazolyl, benzo[d]isothiazolyl, benzo[c]isothiazolyl, benzo[d]thiazolyl, indazolyl, benzo[d]imidazolyl, benzo[d]imidazolyl, and benzo[d][1,2,3]triazolyl.

The term “heterocycloalkyl” refers to a monocycloalkyl group, for example a C₃₋₇monocycloalkyl, or a bicycloalkyl group, for example C₅₋₁₂bicycloalkyl, wherein 1-3 of the carbon atoms are replaced with independently selected heteroatoms, such as nitrogen, oxygen, and sulfur (including its oxidation states: S(O) and SO₂), herein referred to as mono₃₋₇heterocycloalkyl and bi₅₋₁₂heterocycloalkyl, respectively. Examples of mono₃₋₇heterocycloalkyl groups include, but are not limited to, aziridinyl, oxiranyl, thiiranyl 1,1-dioxide, oxetanyl, azetidinyl, thietanyl 1,1-dioxide, pyrrolidinyl, tetrahydrofuranyl, piperidinyl, tetrahydro-2H-pyranyl, morpholinyl, thiomorpholinyl, and piperazinyl. Examples of bi₅₋₁₂heterocycloalkyl groups include, but are not limited to, 1,4-dioxaspiro[4.5]decanyl and 1,5-dioxaspiro[5.5]undecanyl.

The terms “hydroxy” and “hydroxyl” as used herein refers to the radical —OH.

The term “hydroxyalkyl” as used herein refers to an alkyl group substituted with one or more hydroxy groups. Examples include, but are not limited to, HOCH₂—, HOCH₂CH₂—, CH₃CH(OH)CH₂— and HOCH₂CH(OH)CH₂—.

The term “hydroxyalkoxy” as used herein refers to an alkoxy group substituted with one or more hydroxy groups. Examples include but are not limited to HOCH₂O—, HOCH₂CH₂O—, CH₃CH(OH)CH₂O— and HOCH₂CH(OH)CH₂O—.

The term “R^{a}R^{b}NC₁₋₆ alkyl-,” as used herein refers to an alkyl group substituted with a R^{a}R^{b}N— group, as defined herein. Examples include but are not limited to NH₂CH₂—, NH(CH₃)CH₂—, N(CH₃)₂CH₂CH₂— and CH₃CH(NH₂)CH₂—.

The term “R^{a}R^{b}NC₁₋₆alkoxy,” as used herein refers to an alkoxy group substituted with a R^{a}R^{b}N— groups, as defined herein. Examples include but are not limited to NH₂CH₂—, NH(CH₃)CH₂O—, N(CH₃)₂CH₂CH₂O—, and CH₃CH(NH₂)CH₂O—.

The term “oxo” as used herein refers to the radical ═O.

As used herein, when a bicyclic ring is shown with a floating point of attachment and/or floating substituents, for example as in

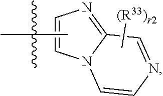

it signifies that the bicyclic ring can be attached via a carbon atom on either ring, and that the substituents (e.g., the R³³ group(s)) can be independently attached to either or both rings.

The terms “Individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HBV infection is desired.

The term “modulation” includes antagonism (e.g., inhibition), agonism, partial antagonism and/or partial agonism.

The term “Pharmaceutically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect.

The term “treating” includes any effect, e.g., lessening, reducing, modulating, or eliminating, a viral infection, that results in the improvement of the disease.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol . . . denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantiomeric and diastereoselective transformations and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255).

### II. Benzothia(dia)zepine Compounds

In one aspect, the present disclosure provides a compound of Formula I

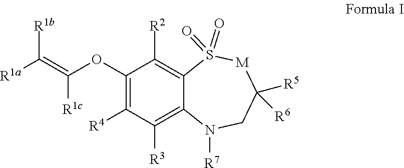

or a pharmaceutically acceptable salt thereof, wherein:
L is a bond, C₁₋₄alkylene or haloC₁₋₄alkylene;
M is NR^{x} or CR^{y}R^{z};

One of R¹ and R^{1a} is R⁰; and the other of R¹ and R^{1a} is selected from the group consisting of hydrogen, halo, C₁₋₄alkyl and haloC₁₋₄alkyl;
R^{1c} is hydrogen or C₁₋₄alkyl;
R⁰ is —C(O)OH, —C(O)OC₁₋₆alkyl, —P(O)(OH)₂, —S(O)₂OH or

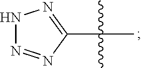 [SMILES: *c1nn[nH]n1]

R², R³ and R⁴ are independently selected from the group consisting of hydrogen, halogen, cyano, C₁₋₄ alkyl, C₃₋₆ cycloalkyl, C₁₋₄ alkoxy, C₃₋₆cycloalkyloxy, C₁₋₄alkylthio, C₃₋₆cycloalkylthio, haloC₁₋₄alkyl, haloC₃₋₆cycloalkyl, haloC₁₋₄alkoxy, haloC₃₋₆cycloalkyloxy, haloC₁₋₄alkylthio, haloC₃₋₆cycloalkylthio, amino, N—(C₁₋₄ alkyl)amino and N,N-di(C₁₋₄ alkyl)amino;
R⁵ and R⁶ are independently selected from the group consisting of hydrogen, C₁₋₆alkyl, C₁₋₆alkenyl, C₁₋₆alkynyl, haloC₁₋₆alkyl, haloC₁₋₆alkenyl, haloC₁₋₆alkynyl, C₁₋₃alkoxyC₁₋₃alkyl, and C₃₋₆cycloalyl-C₁₋₄alkylene-; or R⁵ and R⁶ together with the carbon atom to which they are attached form a C₃₋₇monocycloalkyl or C₅₋₁₂bicycloalkyl, wherein the C₃₋₇monocycloalkyl or C₅₋₁₂bicycloalkyl is optionally substituted with 1-6 substituents independently selected from the group consisting of halogen, CN, NO₂, OH, R^{a}R^{b}N—, C₁₋₄alkyl and haloC₁₋₄alkyl;
R⁷ is -L-R^{7a}, C₁₋₆alkyl, C₂₋₆alkenyl, or C₂₋₆alkynyl, wherein the C₁₋₆alkyl, C₂₋₆alkenyl, or C₂₋₆alkynyl is optionally substituted with 1-6 substituents independently selected from the group consisting of halogen, CN, NO₂, OH and R^{a}R^{b}N—;
R^{7a} is a C₃₋₇monocycloalkyl, C₅₋₁₂bicycloalkyl, mono₃₋₇heterocycloalkyl, bi₅₋₁₂heterocycloalkyl, phenyl, monocyclo₅₋₆heteroaryl or bicyclo₈₋₁₂heteroaryl group, wherein the C₃₋₇monocycloalkyl, C₅₋₁₂bicycloalkyl, mono₃₋₇heterocycloalkyl, bi₅₋₁₂heterocycloalkyl, phenyl, monocyclo₅₋₆heteroaryl or bicyclo₈₋₁₂heteroaryl group is optionally substituted with 1-6 R⁸ groups;
R⁸ is independently selected for each occurrence from the group consisting of halogen, OH, CN, NO₂, hydrazino, formyl, azido, silyl, siloxy, HOC(O)—, R^{a}R^{b}N—, R^{a}R^{b}NS(O)_{q}—, C₁₋₆alkyl, C₂₋₆alkenyl, C₂₋₆alkynyl, haloC₁₋₆alkyl, hydroxyC₁₋₆alkyl-, R^{a}R^{b}NC₁₋₆alkyl-, HOC(O)C₁₋₆alkyl-, R^{a}R^{b}NC₁₋₆alkylNR^{c}—, C₁₋₆alkylNR^{a}C₁₋₆alkylNR^{c}—, C₁₋₆alkoxy, haloC₁₋₆alkoxy, hydroxyC₁₋₆alkoxy-, R^{a}R^{b}NC₁₋₆alkoxy-, C₁₋₆alkoxyC₁₋₆alkyl-, haloC₁₋₆alkoxyC₁₋₆alkyl-, R^{a}R^{b}NC(O)—, C₁₋₆alkylC(O)—, C₁₋₆alkoxyC(O)—, C₁₋₆alkylC(O)O—, C₁₋₆alkylS(O)_{q}—, C₁₋₆alkylS(O)_{q}NR^{c}—, C₁₋₆alkylS(O)_{q}C₁₋₆alkyl-, C₁₋₆alkylS(O)_{q}NR^{a}C₁₋₆alkyl-, C₃₋₆cycloalkylS(O)_{q}C₁₋₆alkyl-, C₁₋₆alkylC(O)C₁₋₆alkyl-, and C₁₋₆alkylC(O)OC₁₋₆alkyl-;
R^{a}, R^{b} and R^{c} are independently selected for each occurrence from the group consisting of hydrogen, C₁₋₆ alkyl, haloC₁₋₆ alkyl and C₃₋₆monocycloalkyl;
R^{x}, R^{y} and R^{z} are independently selected from the group consisting of hydrogen, halo, CN, NO₂, C₁₋₄alkyl, and C₁₋₄haloC₁₋₄alkyl; and
q is independently selected for each occurrence from the group consisting of 0, 1 and 2;
with the proviso that when R^{7a} is an optionally substituted phenyl, L is C₁₋₄alkylene or haloC₁₋₄alkylene.

The following embodiments further describe a compound of Formula I, or a pharmaceutically acceptable salt thereof. It will be appreciated that all chemically allowable combinations of the embodiments described herein are envisioned as further embodiments of the invention.

In certain embodiments, M is NR^{x}.

In certain embodiments, M is NR^{x}, and R^{x} is H or CH₃.

In certain embodiments, M is NR^{x} and R^{x} is CH₃.

In certain embodiments, M is CR^{y}R^{z}, R^{y} is halo, and R^{z} is halo.

In certain embodiments, M is CR^{y}R^{z}, R^{y} is hydrogen, and R^{z} is halo.

In certain embodiments, R^{a} is —C(O)OH, —C(O)OC₁₋₆alkyl or —S(O)₂OH.

In certain embodiments, R¹ is

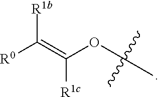

In certain embodiments, R^{1a} is C(O)OH.

In certain embodiments, R^{1a} is S(O)₂OH.

In certain embodiments, R^{1b} is hydrogen or F.

In certain embodiments, R^{1b} is F.

In certain embodiments, R^{1a} is —C(O)OH and R^{1b} is F.

In certain Embodiments, R^{1c} is hydrogen.

In certain Embodiments, R^{1a} is —C(O)OH; R^{1b} is F; and R^{1c} is hydrogen.

In certain embodiments, R² is hydrogen.

In certain embodiments, R³ is hydrogen.

In certain embodiments, R² and R³ are hydrogen.

In certain embodiments, R⁴ is selected from the group consisting of hydrogen, fluoro, chloro, bromo, methyl, cyclopropyl, methoxy, ethoxy, methylthio, ethylthio, amino, methylamino and dimethylamino.

In certain embodiments, R⁴ is methylthio.

In certain embodiments, R⁵ and R⁶ are independently selected from the group consisting of hydrogen and C₁₋₆alkyl.

In certain embodiments, R⁵ is C₁₋₆alkyl and R⁶ is hydrogen.

In certain embodiments, R⁵ is C₃₋₄alkyl and R⁶ is hydrogen.

In certain embodiments, R⁵ is n-butyl and R⁶ is hydrogen.

In certain embodiments, R⁵ and R⁶ are independently selected from the group consisting of hydrogen, C₁₋₆alkenyl, C₁₋₆alkynyl, haloC₁₋₆alkyl, haloC₁₋₆alkenyl, haloC₁₋₆alkynyl, C₁₋₃alkoxyC₁₋₃alkyl, and C₃₋₆cycloalyl-C₁₋₆alkylene-; or R⁵ and R⁶ together with the carbon atom to which they are attached form a C₃₋₇monocycloalkyl or C₅₋₁₂bicycloalkyl, wherein the C₃₋₇monocycloalkyl or C₅₋₁₂bicycloalkyl is optionally substituted with 1-3 substituents independently selected from the group consisting of halogen, CN, NO₂, OH, R^{a}R^{b}N—, C₁₋₄alkyl and haloC₁₋₄alkyl.

In certain embodiments, R⁵ and R⁶ are independently selected from the group consisting of hydrogen, C₁₋₆alkenyl, C₁₋₆alkynyl, haloC₁₋₆alkyl, haloC₁₋₆alkenyl, haloC₁₋₆alkynyl, C₁₋₃alkoxyC₁₋₃alkyl, and C₃₋₆cycloalyl-C₁₋₄alkylene-.

In certain embodiments, R⁵ is C₁₋₆alkenyl, C₁₋₆alkynyl, haloC₁₋₆alkyl, haloC₁₋₆alkenyl, haloC₁₋₆alkynyl, C₁₋₃alkoxyC₁₋₃alkyl, or C₃₋₆cycloalyl-C₁₋₄alkylene-; and R⁶ is hydrogen.

In certain embodiments, R⁵ and R⁶ together with the carbon atom to which they are attached form a C₃₋₇monocycloalkyl or C₅₋₁₂bicycloalkyl, wherein the C₃₋₇monocycloalkyl or C₅₋₁₂bicycloalkyl is optionally substituted with 1-3 substituents independently selected from the group consisting of halogen, CN, NO₂, OH, R^{a}R^{b}N—, C₁₋₄alkyl and haloC₁₋₄alkyl.

In certain embodiments, R⁵ and R⁶ together with the carbon atom to which they are attached form a

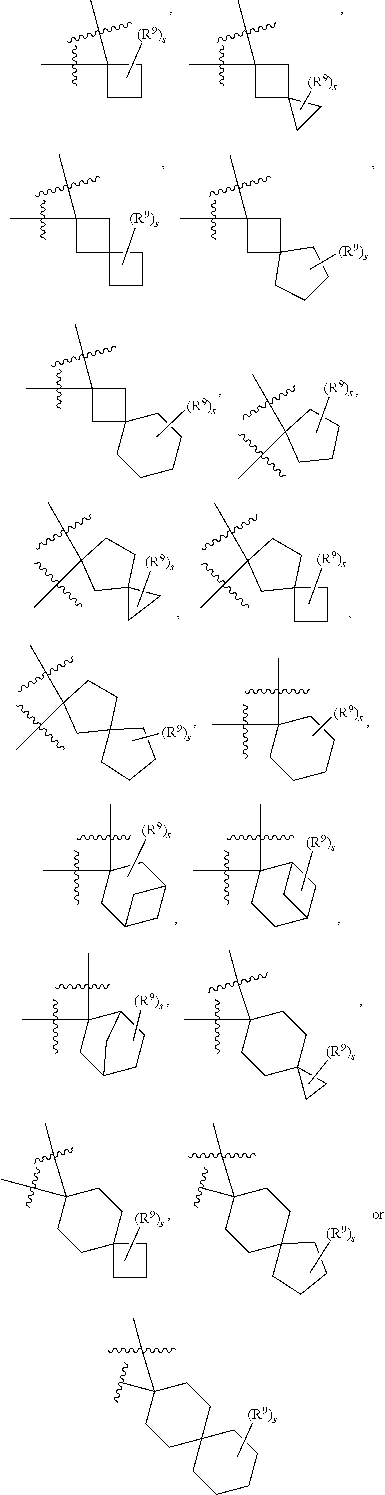

group;
wherein:
R⁹ is independently selected for each occurrence from the group consisting of halogen, CN, NO₂, OH, R^{a}R^{b}N—, C₁₋₄alkyl and haloC₁₋₄alkyl; and
s is selected from the group consisting of 0, 1, 2 and 3.

In certain embodiments, s is 0.

In certain embodiments, R⁷ is C₁₋₆alkyl, C₂₋₆alkenyl, or C₂₋₆alkynyl, wherein the C₁₋₆ alkyl, C₂₋₆alkenyl, or C₂₋₆alkynyl is optionally substituted with 1-4 substituents independently selected from the group consisting of halogen, CN, NO₂, OH and R^{a}R^{b}N—.

In certain embodiments, R⁷ is -L-R⁷; and
R^{7a} is a C₃₋₇monocycloalkyl or C₅₋₁₂bicycloalkyl, wherein the C₃₋₇monocycloalkyl or C₅₋₁₂bicycloalkyl is optionally substituted with 1-6 R⁸ group.

In certain embodiments, R⁷ is -L-R^{7a}; and
R⁷ is a C₃₋₇monocycloalkyl optionally substituted with 1-6 R⁸ group.

In certain embodiments, R⁷ is R^{7a}; and
R^{7a} is a C₃₋₇monocycloalkyl optionally substituted with 1-6 R⁸ group.

In certain embodiments, R⁷ is -L-R^{7a}; and
R^{7a} is a mono₃₋₇heterocycloalkyl, or bi₅₋₁₂heterocycloalkyl, wherein the mono₃₋₇heterocycloalkyl, or bi₅₋₁₂heterocycloalkyl is optionally substituted with 1-6 R⁸ groups.

In certain embodiments, R⁷ is -L-R^{7a}; and
R^{7a} is a mono₃₋₇heterocycloalkyl optionally substituted with 1-6 R⁸ groups.

In certain embodiments, R⁷ is R^{7a}; and
R^{7a} is a mono₃₋₇heterocycloalkyl optionally substituted with 1-6 R⁸ groups.

In certain embodiments, R⁷ is -L-R^{7a}; and

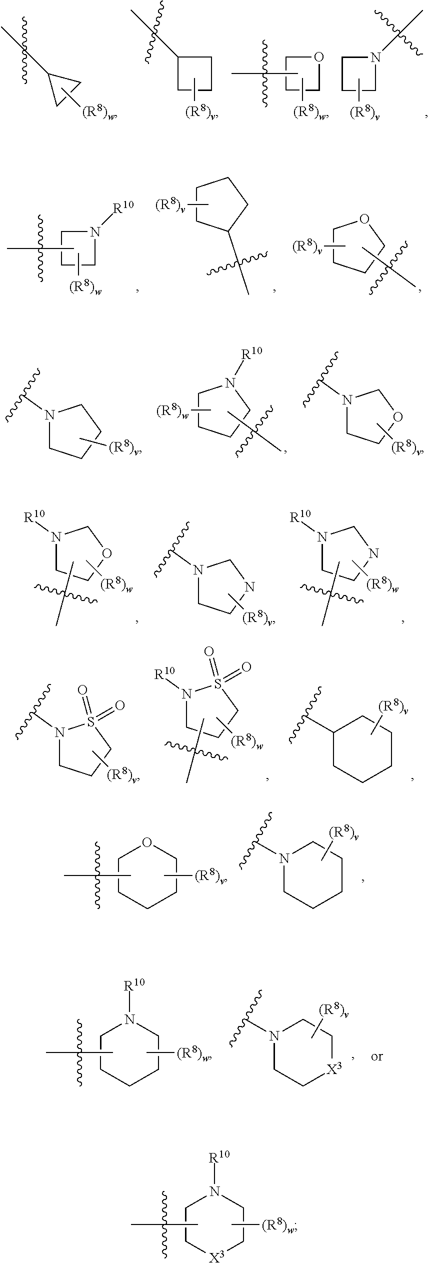

wherein:
R¹⁰ is hydrogen or C₁₋₆alkyl;
X³ is O, S or NR⁸;
v is 0, 1, 2 or 3; and
w is 0, 1 or 2.

In certain embodiments, R⁷ is R^{7a}; and
R^{7a} is

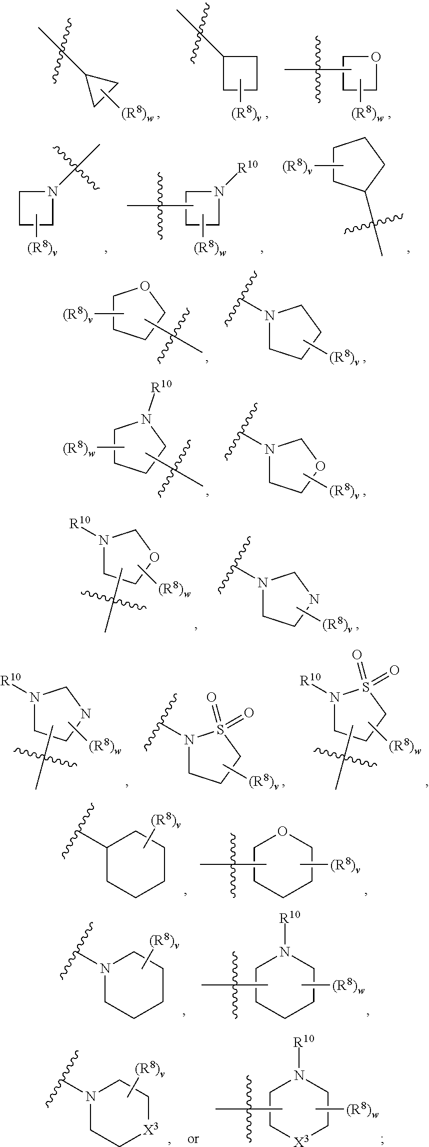

wherein:
R¹⁰ is hydrogen or C₁₋₆alkyl;
X³ is O, S or NR⁸;
v is 0, 1, 2 or 3; and
w is 0, 1 or 2.

In certain embodiments, R⁷ is -L-R^{7a};
L is C₁₋₄alkylene or haloC₁₋₄alkylene; and
R^{7a} is a phenyl optionally substituted with 1-6 R⁸ groups.

In certain embodiments, R⁷ is -L-R^{7a}; and R^{7a} is a phenyl optionally substituted with 1-6 R⁸ groups.

In certain embodiments, R⁷ is benzyl optionally substituted with 1-6 R⁸ groups.

In certain embodiments, R⁷ is -L-R^{7a}; and
R^{7a} is a monocyclo₅₋₆heteroaryl or bicyclo₈₋₁₂heteroaryl group, wherein the monocyclo₅₋₆heteroaryl or bicyclo₈₋₁₂heteroaryl group is optionally substituted with 1-6 R⁸ groups.

In certain embodiments, R⁷ is -L-R^{7a}; and
R^{7a} is a monocyclo₅₋₆heteroaryl optionally substituted with 1-6 R⁸ groups.

In certain embodiments, R⁷ is R^{7a}; and
R^{7a} is a bicyclo₈₋₁₂heteroaryl optionally substituted with 1-6 R⁸ groups.

In certain embodiments, R⁷ is -L-R^{7a};
R^{7a} is

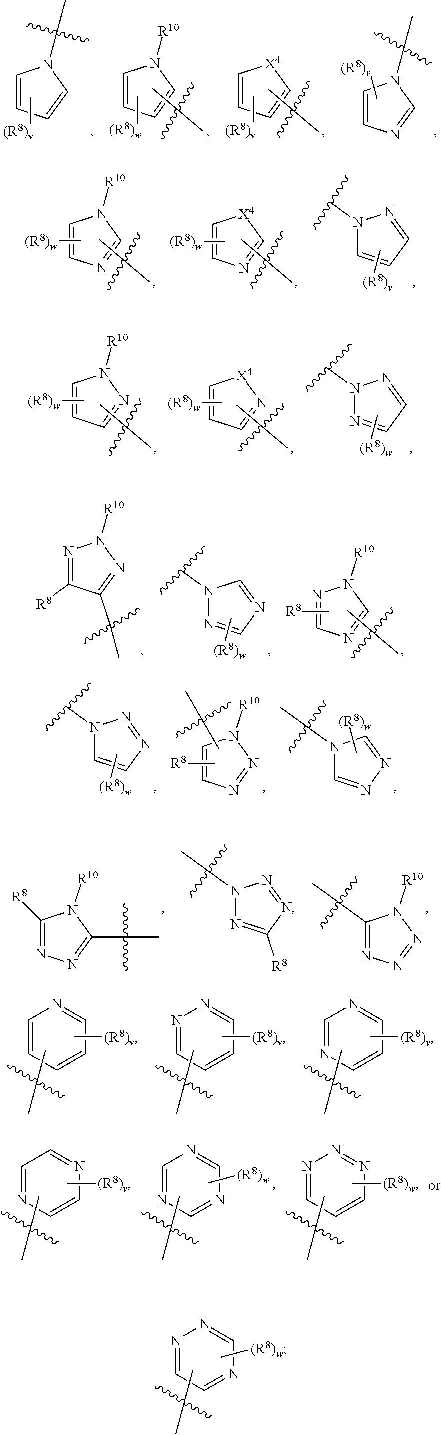

R¹⁰ is hydrogen or C₁₋₆alkyl;
X⁴ is O or S;
v is 0, 1, 2 or 3; and
w is 0, 1 or 2.

In certain embodiments, R⁷ is R^{7a};
R^{7a} is

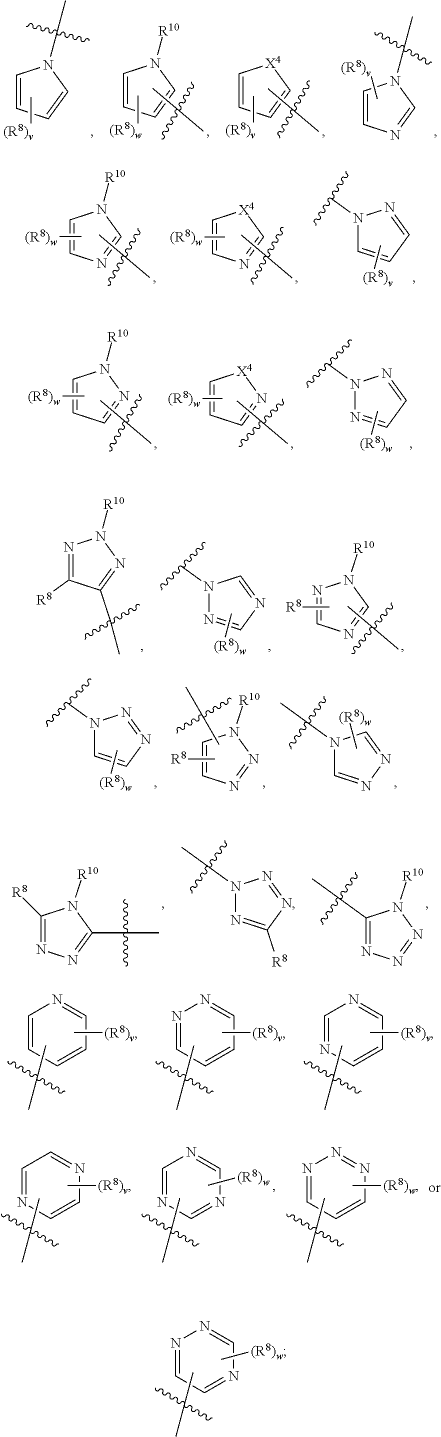

R¹⁰ is hydrogen or C₁₋₆alkyl;
X⁴ is O or S;
v is 0, 1, 2 or 3; and
w is 0, 1 or 2.

In another aspect, the present disclosure provides a compound of Formula II

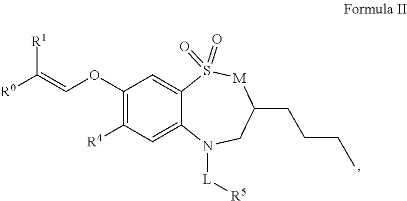

or a pharmaceutically acceptable salt thereof, wherein:
L is C₁₋₄alkylene or C(O);
M is NR^{x} or CR²R³;
R^{x} is hydrogen or C₁₋₄alkyl;
R⁰ is —C(O)OH, —C(O)OC₁₋₆alkyl, or —S(O)₂OH;
R¹ is selected from the group consisting of hydrogen, halo, C₁₋₄alkyl and haloC₁₋₄alkyl;
R² and R³ are independently selected from the group consisting of hydrogen, halo and methyl;
R⁴ is independently selected from the group consisting of hydrogen, halo, cyano, C₁₋₄alkyl, haloC₁₋₄alkyl, C₁₋₄alkoxy and C₁₋₄alkylthio;
R⁵ is an C₁₋₆alkyl, C₂₋₆alkenyl, C₂₋₆alkynyl, haloC₁₋₆alkyl, C₃₋₇monocycloalkyl, -L-C₃₋₇ monocycloalkyl, mono₃₋₇heterocycloalkyl, -L-mono₃₋₇heterocycloalkyl, -L-phenyl, monocyclo₅₋₆heteroaryl or -L-monocyclo₅₋₆heteroaryl group, wherein the C₃₋₇monocycloalkyl, -L-C₃₋₇monocycloalkyl, mono₃₋₇heterocycloalkyl, -L-mono₃₋₇heterocycloalkyl, -L-phenyl, monocyclo₅₋₆heteroaryl or -L-monocyclo₅₋₆heteroaryl group is optionally substituted with 1-3 substituents independently selected from the group consisting of halo, OH, CN, C₁₋₄alkyl, haloC₁₋₄alkyl, C₁₋₄alkoxy and C₁₋₄alkylthio.

The following embodiments further describe a compound of Formula II, or a pharmaceutically acceptable salt thereof. It will be appreciated that all chemically allowable combinations of the embodiments described herein are envisioned as further embodiments of the invention.

In certain embodiments, M is NR^{x}.

In certain embodiments, M is NH or NCH₃.

In certain embodiments, M is CH₂, CF or CF₂.

In certain embodiments, R^{a} is —C(O)OH or —C(O)OC₁₋₄alkyl.

In certain embodiments, R^{a} is —C(O)OH.

In certain embodiments, Ra is S(O)₂OH.

In certain embodiments, R¹ is hydrogen or F.

In certain embodiments, R¹ is F.

In certain embodiments, R⁰ is —C(O)OH or —C(O)OC₁₋₄alkyl and R¹ is hydrogen or F.

In certain embodiments, R⁰ is —C(O)OH and R¹ is F.

In certain embodiments, R⁴ is methylthio.

In certain embodiments, R⁵ is an C₁₋₆alkyl, C₂₋₆alkenyl, C₂₋₆alkynyl or haloC₁₋₆alkyl group.

In certain embodiments, R⁵ is C₃₋₇monocycloalkyl, -L-C₃₋₇monocycloalkyl, mono₃₋₇heterocycloalkyl or -L-mono₃₋₇heterocycloalkyl group, wherein the C₃₋₇monocycloalkyl, -L-C₃₋₇monocycloalkyl, mono₃₋₇heterocycloalkyl or -L-mono₃₋₇heterocycloalkyl group is optionally substituted with 1-3 substituents independently selected from the group consisting of halo, OH, CN, C₁₋₄alkyl, haloC₁₋₄alkyl, C₁₋₄alkoxy and C₁₋₄alkylthio.

In certain embodiments, R⁵ is C₃₋₇monocycloalkyl or mono₃₋₇heterocycloalkyl group, wherein the C₃₋₇monocycloalkyl or mono₃₋₇heterocycloalkyl group is optionally substituted with 1-3 substituents independently selected from the group consisting of halo, OH, CN, C₁₋₄alkyl, haloC₁₋₄alkyl, C₁₋₄alkoxy and C₁₋₄alkylthio.

In certain embodiments, R⁵ is C₃₋₇monocycloalkyl group optionally substituted with 1-3 substituents independently selected from the group consisting of halo, OH, CN, C₁₋₄alkyl, haloC₁₋₄alkyl, C₁₋₄alkoxy and C₁₋₄alkylthio.

In certain embodiments, R⁵ is a mono₃₋₇heterocycloalkyl group optionally substituted with 1-3 substituents independently selected from the group consisting of halo, OH, CN, C₁₋₄alkyl, haloC₁₋₄alkyl, C₁₋₄alkoxy C₁₋₄alkylthio.

In certain embodiments, R⁵ is selected from the group consisting of

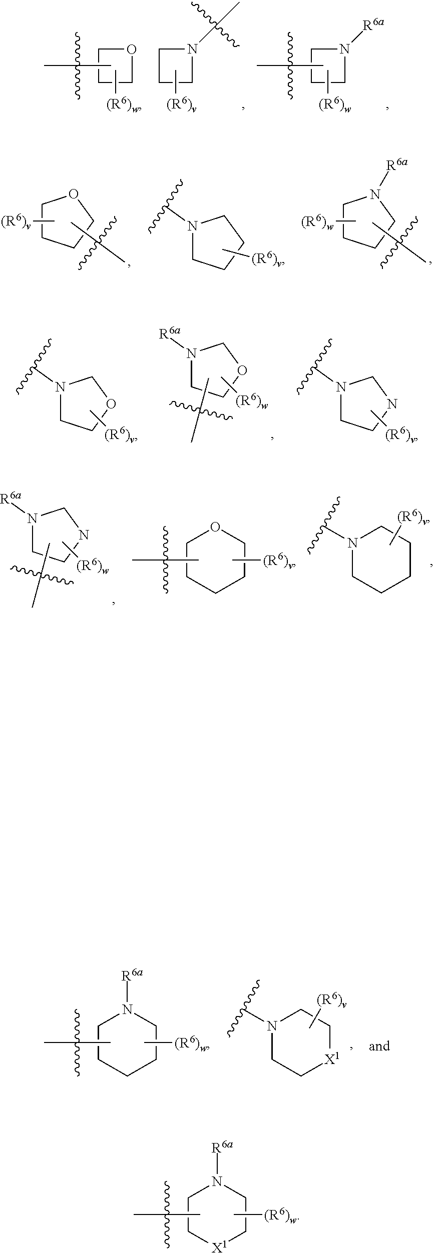

wherein:
R⁶ is independently selected for each occurrence from the group consisting of halo, OH, CN, C₁₋₄alkyl, haloC₁₋₄alkyl, C₁₋₄alkoxy and C₁₋₄alkylthio;
R^{6a} is hydrogen or C₁₋₆alkyl;
X¹ is O, S or NR⁶;
v is 0, 1, 2 or 3; and
w is 0, 1 or 2.

In certain embodiments, R⁵ is a -L-phenyl group optionally substituted with 1-3 substituents independently selected from the group consisting of halo, OH, CN, C₁₋₄alkyl, haloC₁₋₄alkyl, C₁₋₄alkoxy and C₁₋₄alkylthio.

In certain embodiments, R⁵ is monocyclo₅₋₆heteroaryl or -L-monocyclo₅₋₆heteroaryl group, wherein the monocyclo₅₋₆heteroaryl or -L-monocyclo₅₋₆heteroaryl group is optionally substituted with 1-3 substituents independently selected from the group consisting of halo, OH, CN, C₁₋₄alkyl, haloC₁₋₄alkyl, C₁₋₄alkoxy and C₁₋₄alkylthio.

In certain embodiments, R⁵ is monocyclo₅₋₆heteroaryl optionally substituted with 1-3 substituents independently selected from the group consisting of halo, OH, CN, C₁₋₄alkyl, haloC₁₋₄alkyl, C₁₋₄alkoxy and C₁₋₄alkylthio.

In certain embodiments, R⁵ is

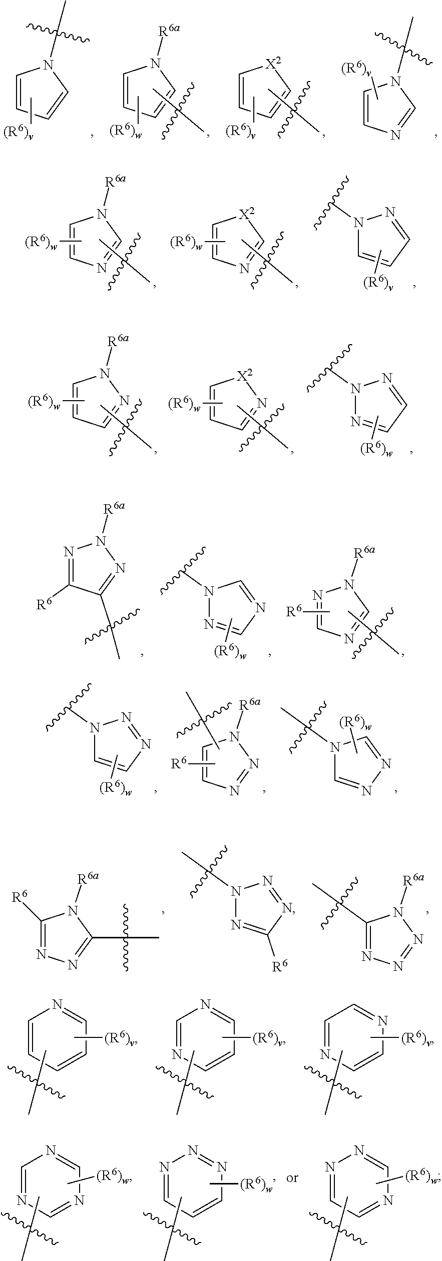

R⁶ is independently selected for each occurrence from the group consisting of halo, OH, CN, C₁₋₄alkyl, haloC₁₋₄alkyl, C₁₋₄alkoxy and C₁₋₄alkylthio;
R^{6a} is hydrogen or C₁₋₆alkyl;
X² is O or S;
v is 0, 1, 2 or 3; and
w is 0, 1 or 2.

### III. Pharmaceutical Compositions and Kits

In another aspect, the disclosure provides pharmaceutical compositions comprising a compound of Formula I, or a pharmaceutically acceptable salt thereof, and a pharmaceutically acceptable excipient. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

In another aspect, the disclosure provides a pharmaceutical composition comprises a compound according to any combination of the Examples described herein, or a pharmaceutically acceptable salt and/or stereoisomer thereof.

Exemplary pharmaceutical compositions of this disclosure may be used in the form of a pharmaceutical preparation, for example, in solid, semisolid or liquid form, which contains one or more compounds of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by e.g., a consumer in need of HBV infection treatment. Such kits include a suitable dosage form such as those described above and instructions describing the method of using such dosage form to mediate, reduce or prevent HBV infection. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### IV. Methods

In a further aspect, a method for treating a hepatitis B infection in a patient in need thereof is provided, comprising administering to a subject or patient an effective amount of a disclosed compound, and/or administering a first disclosed compound and optionally, an additional, different disclosed compound(s). In another embodiment, a method for treating a hepatitis B infection in a patient in need thereof is provided, comprising administering to a subject or patient a therapeutically effective amount of a disclosed pharmaceutical composition or a pharmaceutical composition comprising a disclosed compound, or two or more disclosed compounds, and a pharmaceutically acceptable excipient.

With regard to HBV/HDV coinfection, HDV encodes HDAg, the HDV protein responsible for HDV RNA replication. HDV infection is facilitated by the interaction of HDAg with HBV viral envelope protein HBsAg, for both entry into the hepatocytes and assembly and release of the HDV virions. See for example, Negro, Cold Spring Harb Perspect Med. 2014 Nov. 3; 4(11):a021550. doi: 10.1101/cshperspect.a021550, herein incorporated by reference with regard to such background teaching. Thus, because HDV infection is dependent on the presence of an existing HBV infection, strategies for treating HBV/HDV coinfection may focus on targeting HBV alone, HDV alone or both viruses together.

Thus, the present disclosure also contemplates a method of treating an HBV or HDV infection, or HBV/HDV coinfection, in a patient in need thereof, comprising administering to a subject or patient an effective amount of a disclosed compound, and/or administering a first disclosed compound and optionally, an additional, different disclosed compound(s). In another embodiment, a method for treating an HBV or HDV infection or HBV/HDV coinfection in a patient in need thereof is provided, comprising administering to a subject or patient a therapeutically effective amount of a disclosed pharmaceutical composition or a pharmaceutical composition comprising a disclosed compound, or two or more disclosed compounds, and a pharmaceutically acceptable excipient.

Without being bound by any theory, methods of treatment may be facilitated by various mechanisms of action. One possibility for treatment involves targeting machinery involved in viral particle assembly. In the case of HBV, inhibiting assembly of the HBV envelope or core by targeting HBsAg would disrupt assembly of the HBV particles. A second strategy would be to inhibit viral replication of HBV and/or HDV. Existing antiviral therapies may apply this approach in the form of replication inhibitors that target, for example, a specific viral RNA polymerase.

Thus, another aspect of the disclosure is a method for inhibiting HBV or HDV viral replication in a patient in need thereof, comprising administering to a subject or patient an effective amount of a disclosed compound, and/or administering a first disclosed compound and optionally, an additional, different disclosed compound(s). In another embodiment, a method for inhibiting HBV or HDV viral replication in a patient in need thereof is provided, comprising administering to a subject or patient a therapeutically effective amount of a disclosed pharmaceutical composition or a pharmaceutical composition comprising a disclosed compound, or two or more disclosed compounds, and a pharmaceutically acceptable excipient.

Methods of treatment may further include targeting the network of bile acid transport proteins that are believed to be the “gateway” of entry for HBV or HDV infection into the hepatocyte. See for example, Slijepcevic et al., Digestive Diseases, 2017; 35:251-258, herein incorporated by reference with regard to such background teaching. The bile acid transport system comprising the sodium taurocholate co-transporting polypeptide (NTCP) and apical sodium dependent bile acid transporter (ASBT) are a set of receptors that ensure effective bile acid transport between the ileum and hepatocyte. HBV/HDV coinfection of hepatocytes is believed to be mediated via the NTCP receptor, making it a possible target for treatment. Without being bound by any theory, an “entry inhibitor” may target any of the possible bile acid transport receptors, including, but not limited to the sodium taurocholate co-transporting polypeptide (NTCP) and apical sodium dependent bile acid transporter (ASBT) to prevent entry of either HBV or HDV virus into the cells. Such entry inhibitors may target all or a portion of the transport receptors to inhibit viral entry.

Thus, another aspect of the disclosure is a method of inhibiting viral entry in hepatocytes in a patient in need thereof, comprising administering to a subject or patient an effective amount of a disclosed compound, and/or administering a first disclosed compound and optionally, an additional, different disclosed compound(s). In another embodiment, a method inhibiting viral entry in hepatocytes in a patient in need thereof is provided, comprising administering to a subject or patient a therapeutically effective amount of a disclosed pharmaceutical composition or a pharmaceutical composition comprising a disclosed compound, or two or more disclosed compounds, and a pharmaceutically acceptable excipient.

Regardless of the mechanism targeted, treatment for patients dealing with HBV or HDV infection or HBV/HDV coinfection may be measured by seroconversion of any of the viral antigens, including but not limited to HBsAg or HBeAg, or maintenance of undetectable levels of these antigens.

For use in accordance with the aspects described herein, the appropriate dosage of the compounds described herein is expected to vary depending on, for example, the particular compound employed, the mode of administration, and the nature and severity of the infection to be treated as well as the specific infection to be treated and is within the purview of the treating physician. Usually, an indicated administration dose may be in the range between about 0.1 to about 1000 g/kg body weight. In some cases, the administration dose of the compound may be less than 400 μg/kg body weight. In other cases, the administration dose may be less than 200 μg/kg body weight. In yet other cases, the administration dose may be in the range between about 0.1 to about 100 μg/kg body weight. The dose may be conveniently administered once daily, or in divided doses up to, for example, four times a day or in sustained release form.

A compound of the present disclosure may be administered by any conventional route, in particular: enterally, topically, orally, nasally, e.g., in the form of tablets or capsules, via suppositories, or parenterally, e.g., in the form of injectable solutions or suspensions, for intravenous, intra-muscular, sub-cutaneous, or intra-peritoneal injection. Suitable formulations and pharmaceutical compositions will include those formulated in a conventional manner using one or more physiologically acceptable carriers or excipients, and any of those known and commercially available and currently employed in the clinical setting. Thus, the compounds may be formulated for oral, buccal, topical, parenteral, rectal or transdermal administration or in a form suitable for administration by inhalation or insufflation (either orally or nasally).

For oral administration, pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g. lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). Preparations may also contain buffer salts, flavoring, coloring and sweetening agents as appropriate.

Preparations for oral administration may also be suitably formulated to give controlled-release or sustained release of the active compound(s) over an extended period. For buccal administration the compositions may take the form of tablets or lozenges formulated in a conventional manner known to the skilled artisan.

A disclosed compound may also be formulated for parenteral administration by injection e.g., by bolus injection or continuous infusion. Formulations for injection may be presented in unit dosage form e.g., in ampoules or in multi-dose containers, with an added preservative. The compositions may take such forms as suspensions, solutions or emulsions in oily or aqueous vehicles, and may contain additives such as suspending, stabilizing and/or dispersing agents. Alternatively, the compound may be in powder form for constitution with a suitable vehicle, e.g., sterile pyrogen-free water, before use. Compounds may also be formulated for rectal administration as suppositories or retention enemas, e.g., containing conventional suppository bases such as cocoa butter or other glycerides.

Also contemplated herein are methods and compositions that include a second active agent or administering a second active agent. For example, in addition to being infected with HBV, a subject or patient can further have HBV infection-related co-morbidities, i.e., diseases and other adverse health conditions associated with, exacerbated by, or precipitated by being infected with HBV. Contemplated herein are disclosed compounds in combination with at least one other agent that has previously been shown to treat these HBV-infection-related conditions.

In some cases, a disclosed compound may be administered as part of a combination therapy in conjunction with one or more antivirals. Example antivirals include nucleoside analogs, interferon α, and other assembly effectors, for instance heteroaryldihydropyrimidines (HAPs) such as methyl 4-(2-chloro-4-fluorophenyl)-6-methyl-2-(pyridin-2-yl)-1,4-dihydropyrimidine-5-carboxylate (HAP-1). For example, provided herein is a method of treating a patient suffering from hepatitis B infection comprising administering to the patient a first amount of a disclosed compound and a second amount of an antiviral, or other anti HBV agent, for example a second amount of a second compound selected from the group consisting of: an HBV capsid assembly promoter (for example, GLS4, BAY 41-4109, AT-130, DVR-23 (e.g., as depicted below),

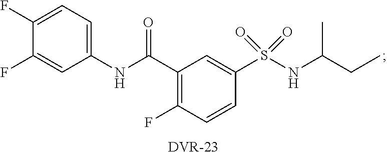

NVR 3-778, NVR1221 (by code); and N890 (as depicted below):

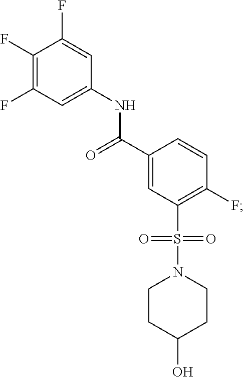 [SMILES: O=C(Nc1cc(F)c(F)c(F)c1)c1ccc(F)c(S(=O)(=O)N2CCC(O)CC2)c1]

other capsid inhibitors such as those disclosed in the following patent applications hereby incorporated by reference: WO2014037480, WO2014184328, WO2013006394, WO2014089296, WO2014106019, WO2013102655, WO2014184350, WO2014184365, WO2014161888, WO2014131847, WO2014033176, WO2014033167, and WO2014033170; Nucleos(t)ide analogs interfering with viral polymerase, such as entecavir (Baraclude), Lamivudine, (Epivir-HBV), Telbivudine (Tyzeka, Sebivo), Adefovir dipivoxil (Hepsera), Tenofovir (Viread), Tenofovir alafenamide fumarate (TAF), prodrugs of tenofavir (e.g. AGX-1009), L-FMAU (Clevudine), LB80380 (Besifovir) and:

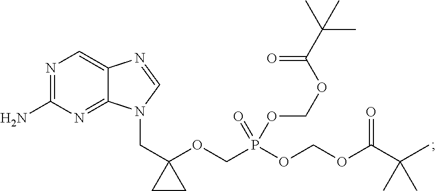 [SMILES: CC(C)(C)C(=O)OCOP(=O)(COC1(Cn2cnc3cnc(N)nc32)CC1)OCOC(=O)C(C)(C)C]

viral entry inhibitors such as Myrcludex B and related lipopeptide derivatives; HBsAg secretion inhibitors such as REP 9AC′ and related nucleic acid-based amphipathic polymers, HBF-0529 (PBHBV-001), PBHBV-2-15 as depicted below:

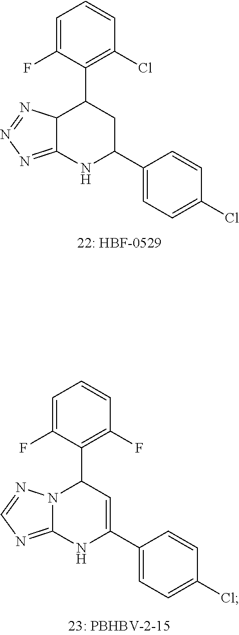 [SMILES: Fc1cccc(Cl)c1C1CC(c2ccc(Cl)cc2)NC2=NN=NC21 ; Fc1cccc(F)c1C1C=C(c2ccc(Cl)cc2)Nc2ncnn21]

and BM601 as depicted below:

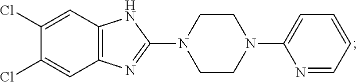 [SMILES: Clc1cc2nc(N3CCN(c4ccccn4)CC3)[nH]c2cc1Cl]

disruptors of nucleocapsid formation or integrity such as NZ-4/W28F:

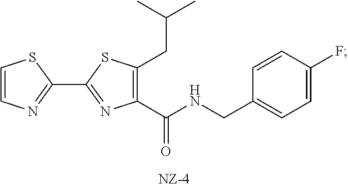 [SMILES: CC(C)Cc1sc(-c2nccs2)nc1C(=O)NCc1ccc(F)cc1]

cccDNA formation inhibitors such as BSBI-25, CCC-0346, CCC-0975 (as depicted below):

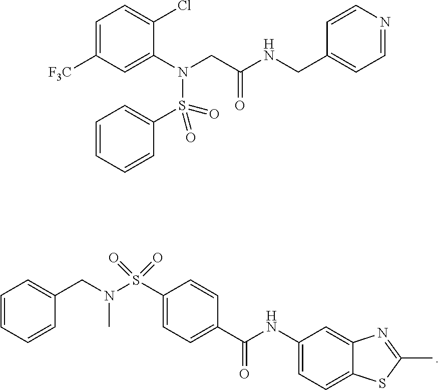 [SMILES: O=C(CN(c1cc(C(F)(F)F)ccc1Cl)S(=O)(=O)c1ccccc1)NCc1ccncc1 ; Cc1nc2cc(NC(=O)c3ccc(S(=O)(=O)N(C)Cc4ccccc4)cc3)ccc2s1]

HBc directed transbodies such as those described in Wang Y, et al, Transbody against hepatitis B virus core protein inhibits hepatitis B virus replication in vitro, Int. Immunopharmacol (2014), located at //dx.doi.org/10.1016/j.intimp.2015.01.028; antiviral core protein mutant (such as Cp183-V124W and related mutations as described in WO/2013/010069, WO2014/074906, each incorporated by reference); inhibitors of HBx-interactions such as RNAi, antisense and nucleic acid based polymers targeting HBV RNA, e.g., RNAi (for example ALN-HBV, ARC-520, TKM-HBV, ddRNAi), antisense (ISIS-HBV), or nucleic acid based polymer: (REP 2139-Ca); immunostimulants such as Interferon alpha 2a (Roferon), Intron A (interferon alpha 2b), Pegasys (peginterferon alpha 2a), Pegylated IFN 2b, IFN lambda 1a and PEG IFN lambda 1a, Wellferon, Roferon, Infergen, lymphotoxin beta agonists such as CBE11 and BS1); Non-Interferon Immune enhancers such as Thymosin alpha-1 (Zadaxin) and Interleukin-7 (CYT107); TLR-7/9 agonists such as GS-9620, CYT003, Resiquimod; Cyclophilin inhibitors such as NVP018; OCB-030; SCY-635; Alisporivir; NIM811 and related cyclosporine analogs; vaccines such as GS-4774, TG1050, Core antigen vaccine; SMAC mimetics such as birinapant and other IAP-antagonists; Epigenetic modulators such as KMT inhibitors (EZH1/2, G9a, SETD7, Suv39 inhibitors), PRMT inhibitors, HDAC inhibitors, SIRT agonists, HAT inhibitors, WD antagonists (e.g. OICR-9429), PARP inhibitors, APE inhibitors, DNMT inhibitors, LSD1 inhibitors, JMJD HDM inhibitors, and Bromodomain antagonists; kinase inhibitors such as TKB1 antagonists, PLK1 inhibitors, SRPK inhibitors, CDK2 inhibitors, ATM & ATR kinase inhibitors; STING Agonists; Ribavirin; N-acetyl cysteine; NOV-205 (BAM205); Nitazoxanide (Alinia), Tizoxanide; SB 9200 Small Molecule Nucleic Acid Hybrid (SMNH); DV-601; Arbidol; FXR agonists (such as GW 4064 and Fexaramin); antibodies, therapeutic proteins, gene therapy, and biologics directed against viral components or interacting host proteins.

In some embodiments, the disclosure provides a method of treating a hepatitis B infection in a patient in need thereof, comprising administering a first compound selected from any one of the disclosed compounds, and one or more other HBV agents each selected from the group consisting of HBV capsid assembly promoters, HBF viral polymerase interfering nucleosides, viral entry inhibitors, HBsAg secretion inhibitors, disruptors of nucleocapsid formation, cccDNA formation inhibitors, antiviral core protein mutant, HBc directed transbodies, RNAi targeting HBV RNA, immunostimulants, TLR-7/9 agonists, cyclophilin inhibitors, HBV vaccines, SMAC mimetics, epigenetic modulators, kinase inhibitors, and STING agonists. In some embodiments, the disclosure provides a method of treating a hepatitis B infection in a patient in need thereof, comprising administering an amount of a disclosed compound, and administering another HBV therapeutic.

In some embodiments, the disclosure further provides a method of treating HBV or HDV infection or HBV/HDV coinfection in a patient in need thereof, comprising administering a first compound selected from any one of the disclosed compounds, and one or more other additional antivirals, the one or more additional antivirals include HDV therapies and one or more of HBV agents each selected from the group consisting of HBV capsid assembly promoters, HBF viral polymerase interfering nucleosides, viral entry inhibitors, HBsAg secretion inhibitors, disruptors of nucleocapsid formation, cccDNA formation inhibitors, antiviral core protein mutant, HBc directed transbodies, RNAi targeting HBV RNA, immunostimulants, TLR-7/9 agonists, cyclophilin inhibitors, HBV vaccines, SMAC mimetics, epigenetic modulators, kinase inhibitors, and STING agonists. In some embodiments, the disclosure provides a method of treating a HBV or HBV infection or HBV/HDV coinfection in a patient in need thereof, comprising administering an amount of a disclosed compound, and administering another HBV therapeutic or an HDV therapeutic.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of a disclosed compound first, for example one or more days or weeks prior to initiation of administration of the antiviral. Moreover, additional drugs may be given in conjunction with the above combination therapy.

In another embodiment, a disclosed compound may be conjugated (e.g., covalently bound directly or through molecular linker to a free carbon, nitrogen (e.g., an amino group), or oxygen (e.g., an active ester) of a disclosed compound), with a detection moiety, for e.g., a fluorophore moiety (such a moiety may for example re-emit a certain light frequency upon binding to a virus and/or upon photon excitation). Contemplated fluorophores include AlexaFluor® 488 (Invitrogen) and BODIPY FL (Invitrogen), as well as fluorescein, rhodamine, cyanine, indocarbocyanine, anthraquinones, fluorescent proteins, aminocoumarin, methoxycoumarin, hydroxycoumarin, Cy2, Cy3, and the like. Such disclosed compounds conjugated to a detection moiety may be used in e.g., a method for detecting HBV or biological pathways of HBV infection, e.g., in vitro or in vivo; and/or methods of assessing new compounds for biological activity.

### V. Examples

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “intermediates” herein are contemplated as compounds of the disclosure.

### Abbreviations

AcOH Acetic acid
ACN Acetonitrile
Boc₂O Di-tert-butyl dicarbonate
nBuLi n-Butyllithium
DCM Dichloromethane
DIAD Diisopropyl azodicarboxylate
DIEA Diisopropyl ethylamine
DMF N,N-Dimethylformamide
DMSO Dimethyl sulfoxide
DPPF 1,1′-Bis(diphenylphosphino)ferrocene
EA, EtOAc Ethyl acetate
Et₃N Triethylamine
HATU Hexafluorophosphate Azabenzotriazole Tetramethyl Uronium
h, hr Hour(s)
HPLC High performance liquid chromatography
LCMS Liquid chromatography-mass spectrometry
MeOH Methanol
NMO N-Methylmoipholine-N-Oxide
NBS N-Bromosuccinimide
PE Petroleum ether
iPrOH Isopropanol
rt, r.t. Room temperature
SFC Supercritical Fluid Chromatography
TEA Triethylamine
TFA Trifluoroacetic acid
THE Tetrahydrofuran
TLC Thin-layer chromatography
XPhos 2-Dicyclohexylphosphino-2′,4′,6′-triisopropylbiphenyl

### Example 1. (E)-3-((3-Butyl-2-methyl-7-(methylthio)-1,1-dioxido-5-(pyridin-4-yl)-2,3,4,5-tetrahydrobenzo[f][1,2,5]thiadiazepin-8-yl)oxy)acrylic acid

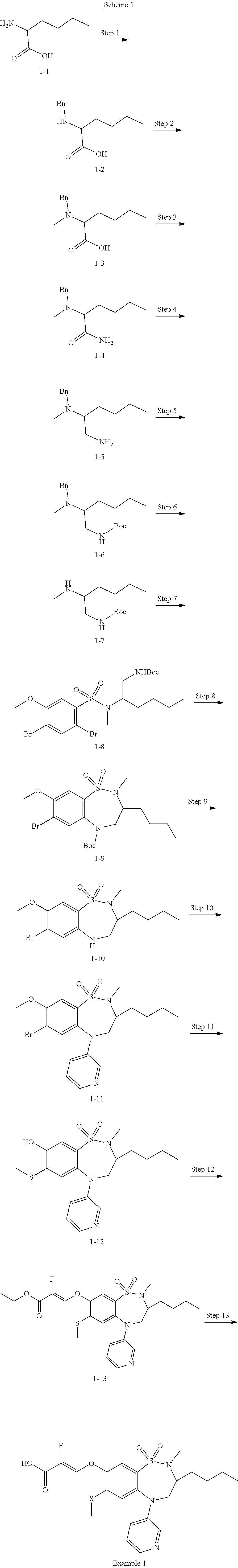

Step 1. Synthesis of 2-(benzylamino)hexanoic acid (1-2). A solution of 2-aminohexanoic acid (1-1) (30 g, 228.7 mmol) and benzaldehyde (24.27 g, 228.7 mmol) in aqueous 2 N aq. NaOH (50 mL) was stirred at 0° C. for 0.5 h, and then NaBH₄ (2.60 g, 68.6 mmol) was added at 0° C. in portions. After stirring at rt for 1 hr, an additional equivalent of benzaldehyde (24.27 g, 228.7 mmol) was added. The resulting mixture was stirred for 0.5 h at 0° C., and then NaBH₄ (2.60 g, 68.6 mmol) was added at 0° C. in portions. After stirring at rt for 2 hr, the reaction mixture was diluted with H₂O (50 mL). The mixture was extracted with DCM (100 mL×2) and then the aqueous layer acidified with 6 N aq. HCl to pH˜6. The precipitated solid was collected through filtration and dried in vacuo to give 1-2 (31 g, 61%) as a white solid. MS (ESI): calcd. for C₃H₁₉NO₂: 221; Found: 222 [M+1]⁺.

Step 2. Synthesis of 2-[benzyl(methyl)amino]hexanoic acid (1-3). A solution of 2-(benzylamino)hexanoic acid (1-2) (31 g, 140.1 mmol) and HCHO (40.92 g, 504.3 mmol) in formic acid (58.03 g, 1260.7 mmol) was stirred at 100° C. for 5 hr. The resulting mixture was concentrated, and the residue was triturated with H₂O (30 mL). The precipitated solid was collected, washed with H₂O (30 mL), dried in vacuo to give 1-3 (20 g, 61%) as a white solid. MS (ESI): calcd. for C₁₄H₂₁NO₂: 235; Found: 236 [M+1]⁺; ¹H NMR: (300 MHz, DMSO-d₆): δ 7.38-7.18 (m, 5H), 3.74 (d, J=13.8 Hz, 1H), 3.60 (d, J=13.8 Hz, 1H), 3.18 (t, J=7.5 Hz, 1H), 2.19 (s, 3H), 1.73-1.63 (m, 1H), 1.61 (dd, J=7.8, 5.4 Hz, 1H), 1.45-1.20 (m, 4H), 0.87 (t, J=6.3 Hz, 3H) ppm.

Step 3. Synthesis of 2-[benzyl(methyl)amino]hexanamide (1-4). To a stirred mixture of 2-[benzyl(methyl)amino]hexanoic acid (1-3) (20 g, 85.0 mmol), DIPEA (21.97 g, 170.0 mmol), and HATU (35.55 g, 93.5 mmol) in DMF (200 mL) was added NH₄Cl (6.82 g, 127.48 mmol) in portions at rt. After stirring at rt for 5 hr, the resulting mixture was diluted with H₂O (200 mL) and extracted with EA (200 mL×2). The combined organic layers were washed with brine (200 mL), dried over anhydrous Na₂SO₄, and concentrated. The residue was purified by silica gel column chromatography using PE/EtOAc=3/1 (v/v) as eluent to afford 1-4 (15 g, 75%) as a yellow oil. MS (ESI): calcd. for C₁₄H₂₂N₂O: 234; Found: 235 [M+1]⁺; ¹H NMR (300 MHz, DMSO-d₆): δ 7.31-7.16 (m, 5H), 6.96 (s, 2H), 3.70-3.56 (m, 2H), 3.51 (d, J=13.8 Hz, 1H), 3.05 (t, J=7.2 Hz, 1H), 2.70 (s, 3H), 1.82-1.71 (m, 1H), 1.39-1.29 (m, 4H), 0.94-0.83 (m, 3H) ppm.

Step 4. Synthesis of (1-aminohexan-2-yl)(benzyl)methylamine (1-5). To a stirred solution of 2-[benzyl(methyl)amino]hexanamide (1-4) (15 g, 64.01 mmol) in THF (100 ml) was added BH₃-Me₂S (12.14 mL, 128.0 mmol) dropwise at 0° C. After stirring at 75° C. for 16 hr, the reaction was quenched with MeOH (50 mL) at 0° C. The resulting mixture was stirred at 70° C. for 2 h and then concentrated under reduced pressure to give 1-5 (13 g, 55%) as a colorless oil, which was used for the next step directly without further purification. MS (ESI): calcd. for C₁₄H₂₄N₂: 222; Found: 223 [M+1]⁺.

Step 5. Synthesis of tert-butyl N-{2-[benzyl(methyl)amino]hexyl}carbamate (1-6). To a stirred solution of (1-aminohexan-2-yl)(benzyl)methylamine (1-5) (13 g, 35.4 mmol) and TEA (10.75 g, 106.2 mmol) in DCM (100 mL) at rt was added Boc₂O (15.45 g, 70.8 mmol) in portions. After stirring at rt for 16 hr, the reaction mixture was diluted with H₂O (100 mL) and extracted with DCM (100 mL×2). The combined organic layers were washed with brine (100 mL), dried over anhydrous Na₂SO₄, and concentrated. The residue was purified by silica gel column chromatography using PE/EtOAc=3/1 (v/v) as eluent to give 1-6 (9 g, 79%) as a colorless oil. MS (ESI): calcd. for C₁₉H₃₂N₂O₂: 320; Found: 321 [M+1]⁺; ¹H NMR (300 MHz, DMSO-d₆): δ 7.29 (d, J=4.5 Hz, 4H), 7.27-7.15 (m, 1H), 6.63 (s, 1H), 3.59 (s, 2H), 3.11 (dt, J=13.2, 6.6 Hz, 1H), 2.96-2.82 (m, 1H), 2.76-2.57 (m, 2H), 2.10 (s, 3H), 1.41 (s, 1H), 1.39 (s, 9H), 1.36-1.16 (m, 2H), 1.09 (t, J=7.2 Hz, 2H), 0.92-0.88 (m, 3H) ppm.

Step 6. Synthesis of tert-butyl N—[2-(methylamino)hexyl]carbamate (1-7). To a solution of tert-butyl N-{2-[benzyl(methyl)amino]hexyl}carbamate (1-6) (9 g, 28.08 mmol) in 100 mL MeOH was added Pd/C (10%, 2 g) under nitrogen atmosphere. After stirring at rt for 16 h under hydrogen atmosphere, the reaction mixture was slushed with N2 and filtered through a Celite® 545 plug. The filtrate was concentrated, and the residue was dried in vacuo to give 1-7 (5 g, 77%) as a colorless oil. MS (ESI): calcd. for C₁₂H₂₆N₂O₂: 230; Found: 231 [M+1]⁺.

Step 7. Synthesis of tert-butyl N—[2-(N-methyl2,4-dibromo-5-methoxybenzenesulfonamido)hexyl]carbamate (1-8). To a stirred solution of tert-butyl N— [2-(methylamino)hexyl]carbamate (1-7) (5 g, 21.7 mmol) and TEA (4.39 g, 43.4 mmol) in THF (100 mL) was added 2,4-dibromo-5-methoxybenzenesulfonyl chloride (7.91 g, 21.7 mmol) in portions at rt. After stirring at rt for 16 hr, the resulting mixture was diluted with H₂O (100 mL) and extracted with EtOAc (100 mL×2). The combined organic layers were washed with brine (100 mL), dried over anhydrous Na₂SO₄, and concentrated. The residue was triturated with PE/EA=1/1 (30 mL). The precipitated solid was washed with PE/EtOAc=1/1 (v/v) (10 mL) and dried in vacuo to give 1-8 (10 g, 83%) as a white solid. MS (ESI): calcd. for C₁₉H₃₀Br₂N₂O₅S: 556; Found: 557 [M+1]⁺; ¹H NMR (300 MHz, DMSO-d₆): δ 7.66-7.55 (m, 1H), 6.58 (t, J=6.0 Hz, 1H), 3.95 (d, J=2.7 Hz, 3H), 3.74-3.61 (m, 2H), 2.97 (q, J=6.3 Hz, 2H), 2.85 (s, 3H), 1.34 (s, 9H), 1.15-1.11 (m, 5H), 0.77 (t, J=6.9 Hz, 3H) ppm.

Step 8. Synthesis of tert-butyl tert-butyl 7-bromo-3-butyl-8-methoxy-2-methyl-3,4-dihydrobenzo[f][1,2,5]thiadiazepine-5(2H)-carboxylate 1,1-dioxide (1-9). A mixture of tert-butyl N—[2-(N-methyl2,4-dibromo-5-methoxybenzenesulfonamido)hexyl]carbamate (1-8) (10 g, 17.91 mmol) and K₂CO₃ (7.43 g, 53.73 mmol), and Cu (5.69 g, 89.55 mmol) in DMF (100 mL) was stirred for 24 h at 115° C. under N₂ atmosphere. The resulting mixture was extracted with EA (100 mL×2). The combined organic layers were washed with brine (100 mL), dried over anhydrous Na₂SO₄, and concentrated. The residue was purified by silica gel column chromatography using PE/THF=4/1 (v/v) as eluent to give 1-9 (6.6 g, 77%) as a brown solid. MS (ESI): calcd. for C₁₉H₂₉BrN₂O₅S: 476; Found: 477 [M+1]⁺; ¹H NMR (300 MHz, DMSO-d₆): δ 7.72 (d, J=8.1 Hz, 1H), 7.44-7.31 (m, 1H), 4.06 (d, J=8.7 Hz, 1H), 3.97 (d, J=2.7 Hz, 2H), 3.80 (dd, J=23.1, 15.0 Hz, 1H), 2.97 (dd, J=25.5, 15.3 Hz, 2H), 2.41 (d, J=3.6 Hz, 2H), 1.45 (s, 3H), 1.37 (s, 9H), 1.28 (s, 4H), 0.97-0.78 (m, 3H) ppm.

Step 9. Synthesis of 7-bromo-3-butyl-8-methoxy-2-methyl-2,3,4,5-tetrahydrobenzo[f][1,2,5]thiadiazepine 1,1-dioxide (1-10). A solution of tert-butyl 7-bromo-3-butyl-8-methoxy-2-methyl-3,4-dihydrobenzo[f][1,2,5]thiadiazepine-5(2H)-carboxylate 1,1-dioxide (1-9) (340 mg, 0.71 mmol) and trifluoroacetic acid (2 mL) in DCM (2 mL) was stirred for 1 hr at rt. The resulting mixture was concentrated under vacuum and the residue was diluted with EA (10 mL). The mixture was basified with sat. aqueous NaHCO₃ solution to pH=8. The resulting mixture was extracted with EtOAc (10 mL×3). The combined organic layers were washed with brine (10 mL), dried over anhydrous Na₂SO₄, and concentrated. The residue was dried n vacuo to give 1-11 (250 mg, 93%) as a yellow solid. MS (ESI): calcd. for C₁₋₄H₂₁BrN₂O₃S: 376; Found: 377 [M+1]⁺.

Step 10. Synthesis of 7-bromo-3-butyl-8-methoxy-2-methyl-5-(pyridin-3-yl)-2,3,4,5-tetrahydrobenzo[f][1,2,5]thiadiazepine 1,1-dioxide (1-11). To a solution of 7-bromo-3-butyl-8-methoxy-2-methyl-2,3,4,5-tetrahydrobenzo[f][1,2,5]thiadiazepine 1,1-dioxide (1-10) (250 mg, 0.66 mmol) and 3-iodopyridine (203.75 mg, 0.99 mmol) in DMF (10 mL) were added Cs₂CO₃ (647.67 mg, 1.98 mmol) and Pd(PPh₃)₄ (38.28 mg, 0.03 mmol) and XPhos (31.59 mg, 0.06 mmol). After stirring at 120° C. for 16 hr under a nitrogen atmosphere, the resulting mixture was extracted with EA (10 mL×3). The combined organic layers were washed with brine (10 mL), dried over anhydrous Na₂SO₄, and concentrated. The residue was purified by silica gel column chromatography using PE/EtOAc=1/1 (v/v) as eluent to give 1-11 (180 mg, 60%) as a brown solid. MS (ESI): calcd. for C₁₉H₂₄BrN₃O₃S: 453; Found: 454 [M+1]⁺.

Step 11. Synthesis of 3-butyl-8-hydroxy-2-methyl-7-(methylthio)-5-(pyridin-3-yl)-2,3,4,5-tetrahydrobenzo[f][1,2,5]thiadiazepine 1,1-dioxide (1-12). A solution of 7-bromo-3-butyl-8-methoxy-2-methyl-5-(pyridin-3-yl)-2,3,4,5-tetrahydrobenzo[f][1,2,5]thiadiazepine 1,1-dioxide (1-12) (180 mg, 0.39 mmol) and (methylsulfanyl)sodium (138.81 mg, 1.98 mmol) in DMF (3 mL) was stirred at 80° C. for 16 h. The resulting mixture was extracted with EA (15 mL×3). The combined organic layers were washed with brine (5 mL), dried over anhydrous Na₂SO₄, and concentrated. The residue was purified by silica gel column chromatography using PE/EtOAc=1/2 (v/v) as eluent to give 1-12 (130 mg, 81%) as a brown solid. MS (ESI): calcd. for C₁₉H₂₅N₃O₃S₂: 407; Found: 408 [M+1]⁺.

Step 12. Synthesis of ethyl (Z)-3-((3-butyl-2-methyl-7-(methylthio)-1,1-dioxido-5-(pyridin-3-yl)-2,3,4,5-tetrahydrobenzo[f][1,2,5]thiadiazepin-8-yl)oxy)-2-fluoroacrylate (1-13). To a solution of ethyl 3-bromo-2,2-difluoropropanoate (127.79 mg, 0.59 mmol) in DMA (3 mL) was added 60% NaH in paraoil (39.26 mg, 0.98 mmol) at 0° C. under an atmosphere of N₂. The mixture was allowed to warm to rt and stirred for 1 hr. Subsequently, 3-butyl-8-hydroxy-2-methyl-7-(methylthio)-5-(pyridin-3-yl)-2,3,4,5-tetrahydrobenzo[f][1,2,5]thiadiazepine 1,1-dioxide (80 mg, 0.20 mmol) in DMA (2 mL) was added. After stirring at 70° C. for 5 hr, the mixture was allowed to cool down to rt and acidified to pH=1 with 1N aq. HCl. The resulting mixture was extracted with EA (10 mL×3). The combined organic layers were washed with brine (10 mL), dried over anhydrous Na₂SO₄, and concentrated. The residue was purified by silica gel column chromatography using PE/EtOAc=1/1 (v/v) as eluent to give ethyl (Z)-3-((3-butyl-2-methyl-7-(methylthio)-1,1-dioxido-5-(pyridin-3-yl)-2,3,4,5-tetrahydrobenzo[f][1,2,5]thiadiazepin-8-yl)oxy)-2-fluoroacrylate (1-13) as a brown solid. MS (ESI): calcd. for C₂₄H₃₀FN₃O₅S₂: 523; Found: 524 [M+1]⁺.

Step 13. Synthesis of (Z)-3-((3-butyl-2-methyl-7-(methylthio)-1,1-dioxido-5-(pyridin-3-yl)-2,3,4,5-tetrahydrobenzo[f][1,2,5]thiadiazepin-8-yl)oxy)-2-fluoroacrylic acid (Example 1). A solution of ethyl (Z)-3-((3-butyl-2-methyl-7-(methylthio)-1,1-dioxido-5-(pyridin-3-yl)-2,3,4,5-tetrahydrobenzo[f][1,2,5]thiadiazepin-8-yl)oxy)-2-fluoroacrylate (1-13) (50 mg, 0.10 mmol) and LiOH (6.86 mg, 0.29 mmol) in dioxane (4 mL) and H₂O (1 mL) was stirred for 2 h at rt. The mixture was acidified to pH=4 with 2N aq. HCl. The resulting mixture was extracted with EtOAc (10 mL×3). The combined organic layers were washed with brine (10 mL), dried over anhydrous Na₂SO₄, and concentrated. The residue (50 mg) was purified by Prep-HPLC with the following conditions: Column, XBridge Shield RP18 OBD Column, 5 um, 19*150 mm; mobile phase, Water (0.05% FA) and ACN (45% PhaseB up to 75% in 8 min); Detector, UV 254 nm) to give Example 1 (15 mg, 32%) as a white solid. MS (ESI): calcd. for C₂₂H₂₆FN₃O₅S₂: 495, Found: 496 [M+1]⁺; ¹H NMR (300 MHz, DMSO-d₆): δ 7.96 (s, 2H), 7.60 (d, J=18.0 Hz, 1H), 7.54 (d, J=12.0 Hz, 1H), 7.26 (s, 1H), 7.17 (s, 1H), 6.99 (d, J=8.4 Hz, 1H), 4.10 (d, J=15.9 Hz, 1H), 3.91-3.89 (m, 1H), 3.08-2.96 (m, 1H), 2.51 (s, 3H), 2.44 (s, 3H), 1.72-1.61 (m, 1H), 1.53-1.49 (m, 1H), 1.48-1.35 (m, 4H), 0.94-0.85 (m, 3H) ppm.

### Example 2. (Z)-3-((3-butyl-5-(4,4-difluorocyclohexyl)-2-methyl-7-(methylthio)-1,1-dioxido-2,3,4,5-tetrahydrobenzo[f][1,2,5]thiadiazepin-8-yl)oxy)-2-fluoroacrylic acid

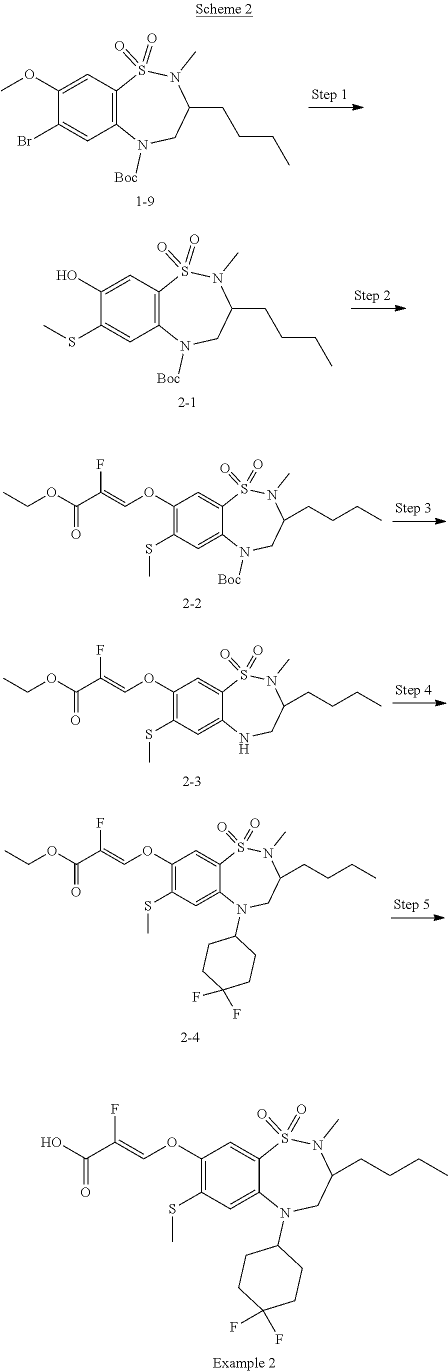

Step 1. Synthesis of tert-butyl 3-butyl-8-hydroxy-2-methyl-7-(methylthio)-3,4-dihydrobenzo[f][1,2,5]thiadiazepine-5(2H)-carboxylate 1,1-dioxide (2-1). A solution of tert-butyl 7-bromo-3-butyl-8-methoxy-2-methyl-3,4-dihydrobenzo[f][1,2,5]thiadiazepine-5(2H)-carboxylate 1,1-dioxide (1-9) (6.0 g, 12.6 mmol) and (methylsulfanyl)sodium (4.4 g, 62.85 mmol) in dimethylformamide (100 mL) was stirred for 16 hr under an atmosphere of N₂. The resulting mixture was extracted with EtOAc (100 mL×3). The combined organic layers were washed with brine (150 mL), dried over anhydrous Na₂SO₄, and concentrated. The residue was purified by silica gel column chromatography using PE/EtOAc=2/1 (v/v) as eluent to give 2-1 (5.0 g, 92%) as a brown solid. MS (ESI): calcd. for C₁₉H₃₀N₂O₅S₂: 430; Found: 431 [M+1]⁺.

Step 2. Synthesis of tert-butyl (Z)-3-butyl-8-((3-ethoxy-2-fluoro-3-oxoprop-1-en-1-yl)oxy)-2-methyl-7-(methylthio)-3,4-dihydrobenzo[f][1,2,5]thiadiazepine-5(2H)-carboxylate 1,1-dioxide (2-2). To a solution of ethyl 3-bromo-2,2-difluoropropanoate (3.0 g, 14.0 mmol) in DMA (60 mL) was added 60% NaH in paraoil (930 mg, 2.3 mmol) at 0° C. under an atmosphere of N₂. The mixture was allowed to warm to rt and stirred for 1 hr. tert-Butyl 3-butyl-8-hydroxy-2-methyl-7-(methylthio)-3,4-dihydrobenzo[f][1,2,5]thiadiazepine-5(2H)-carboxylate 1,1-dioxide (2-1) (2.0 g, 4.64 mmol) in DMA (20 mL) was added. After stirring at 70° C. for 5 hr, the mixture was allowed to cool down to rt and acidified to pH=1 with 1N aq. HCl solution. The resulting mixture was extracted with EA (100 mL×2). The combined organic layers were washed with brine (100 mL), dried over anhydrous Na₂SO₄, and concentrated. The residue was purified by silica gel column chromatography using PE/EtOAc=3/1 (v/v) to give 2-2 (1.4 g, 55%) as a brown solid. MS (ESI): calcd. for C₂₄H₃₅FN₂O₇S₂: 546; Found: 547 [M+1]⁺.

Step 3. Synthesis of ethyl (Z)-3-((3-butyl-2-methyl-7-(methylthio)-1,1-dioxido-2,3,4,5-tetrahydrobenzo[f][1,2,5]thiadiazepin-8-yl)oxy)-2-fluoroacrylate (2-3). To a stirred solution of tert-butyl (Z)-3-butyl-8-((3-ethoxy-2-fluoro-3-oxoprop-1-en-1-yl)oxy)-2-methyl-7-(methylthio)-3,4-dihydrobenzo[f][1,2,5]thiadiazepine-5(2H)-carboxylate 1,1-dioxide (2-2) (1.4 g, 2.56 mmol) in DCM (20 mL) was added TFA (20 mL). After stirring at rt for 5 hr, the resulting mixture was concentrated. The residue was diluted with EA (10 mL) and adjusted pH to ˜8 using sat aq. Na₂CO₃. The resulting mixture was extracted with EA (25 mL×3). The combined organic layers were washed with brine (25 mL), dried over anhydrous Na₂SO₄, and concentrated. The residue was dried in vacuo to give crude 2-3 (1.0 g, 87%) as a yellow solid, which was used for the next step without further purification. MS (ESI): calcd. for C₁₉H₂₇FN₂O₅S₂: 446; Found: 447 [M+1]⁺.

Step 4. Synthesis of ethyl (Z)-3-((3-butyl-5-(4,4-difluorocyclohexyl)-2-methyl-7-(methylthio)-1,1-dioxido-2,3,4,5-tetrahydrobenzo[f][1,2,5]thiadiazepin-8-yl)oxy)-2-fluoroacrylate (2-4). To a stirred solution of ethyl (Z)-3-((3-butyl-2-methyl-7-(methylthio)-1,1-dioxido-2,3,4,5-tetrahydrobenzo[f][1,2,5]thiadiazepin-8-yl)oxy)-2-fluoroacrylate (2-3) (50 mg, 0.11 mmol) and 4,4-difluorocyclohexan-1-one (45.05 mg, 0.34 mmol) in tetrahydrofuran (10 mL) was added phenylsilane (36.35 mg, 0.34 mmol) and dibutyldichlorostannane (51.03 mg, 0.17 mmol) at rt. After stirring at 60° C. for 72 h, the mixture was allowed to cool down to rt and concentrated. The residue was purified by Prep-TLC (PE/EtOAc=2/1 (v/v)) to give 2-4 (35 mg, 55%) as a brown solid. MS (ESI): calcd. for C₂₅H₃₅F₃N₂O₅S₂: 564; Found: 565 [M+1]⁺.

Step 5. Synthesis of ((Z)-3-((3-butyl-5-(4,4-difluorocyclohexyl)-2-methyl-7-(methylthio)-1,1-dioxido-2,3,4,5-tetrahydrobenzo[f][1,2,5]thiadiazepin-8-yl)oxy)-2-fluoroacrylic acid (Example 2). A solution of ethyl (Z)-3-((3-butyl-5-(4,4-difluorocyclohexyl)-2-methyl-7-(methylthio)-1,1-dioxido-2,3,4,5-tetrahydrobenzo[f][1,2,5]thiadiazepin-8-yl)oxy)-2-fluoroacrylate (2-4) (35 mg, 0.06 mmol) and LiOH (4.45 mg, 0.19 mmol) in dioxane (4 mL) and H₂O (1 mL) was stirred for 2 h at it. The resulting mixture was diluted with water (10 mL), acidified to pH=4 with 0.1 N aq. HCl solution, and extracted with EtOAc (10 mL×3). The combined organic layers were washed with brine (10 mL), dried over anhydrous Na₂SO₄, and concentrated. The residue was purified by Prep-HPLC with the following conditions: Column, XBridge Shield RP18 OBD Column, 5 um, 19*150 mm; mobile phase, Water (0.05% FA) and ACN (45% PhaseB up to 75% in 7 min); Detector: UV 254 nm) to give Example 2 (8 mg, 24%) as a white solid. MS (ESI): calcd. for C₂₃H₃₁F₃N₂OS₂: 536; Found: 537 [M+1]⁺; ¹H NMR (300 MHz, DMSO-d6): δ 7.44 (s, 1H), 7.36 (d, J=5.4 Hz, 1H), 6.82 (s, 1H), 3.80 (s, 1H), 3.51 (s, 1H), 3.33-3.31 (m, 21H), 2.74-2.60 (m, 2H), 2.54 (s, 3H), 2.28-1.93 (m, 6H), 1.90-1.70 (m, 2H), 1.51-1.46 (m, 2H), 1.36-1.30 (m, 4H), 1.26-1.23 (m, 1H), 0.97-0.87 (m, 3H) ppm.

Table 1 shows structures and analytical data for representative Examples of the present invention. These compounds can be prepared according to the synthetic schemes described above and using procedures known to those of ordinary skill in the art.

TABLE 1
Representative Examples of the present invention

| Example | Structure | MS | ¹H NMR |
|---|---|---|---|
| 2a | 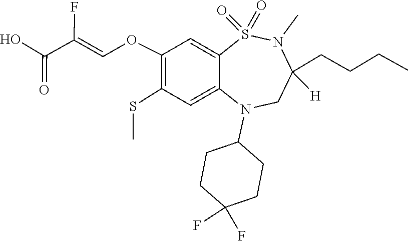 [SMILES: CCCCC1CN(C2CCC(F)(F)CC2)c2cc(SC)c(O/C=C(\F)C(=O)O)cc2S(=O)(=O)N1C] Single enantiomer I | 538 [M + 1]⁺ | ¹H NMR (300 MHz, DMSO-d6): δ 7.42-7.35 (m, 2H), 6.81 (s, 1H), 3.88-3.73 (m, 1H), 3.59-3.43 (m, 1H), 3.35-3.30 (m, 1H), 2.65-2.58 (m, 2H), 2.54 (s, 3H), 2.51 (s, 3H), 2.20-2.08 (m, 3H), 1.96-1.88 (m, 2H), 1.85-1.72 (m, 2H), 1.55-1.45 (m, 2H), 1.40-1.30 (m, 4H), 1.00- 0.89 (m, 3H) ppm |
| 2b | 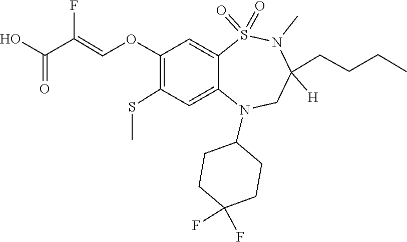 [SMILES: CCCCC1CN(C2CCC(F)(F)CC2)c2cc(SC)c(O/C=C(\F)C(=O)O)cc2S(=O)(=O)N1C] Single enantiomer II | 538 [M + 1]⁺ | ¹H NMR (300 MHz, DMSO-d6): δ 7.42-7.35 (m, 2H), 6.81 (s, 1H), 3.80-3.74 (m, 1H), 3.57-3.51 (m, 1H), 3.34-3.30 (m, 1H), 2.62-2.58 (m, 2H), 2.54 (s, 3H), 2.51 (s, 3H), 2.27-2.08 (m, 3H), 2.00-1.93 (m, 2H), 1.78-1.76 (m, 2H), 1.55-1.48 (m, 2H), 1.40-1.30 (m, 4H), 0.93- 0.89 (m, 3H) ppm |
| 3 | 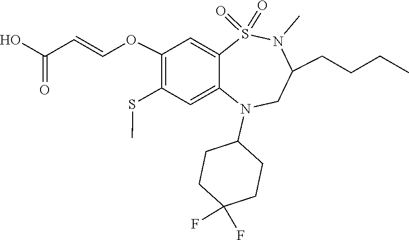 [SMILES: CCCCC1CN(C2CCC(F)(F)CC2)c2cc(SC)c(O/C=C/C(=O)O)cc2S(=O)(=O)N1C] A mixture of two enantiomers | 519 [M + 1]⁺ | ¹H NMR (300 MHz, DMSO-d6): δ 12.11 (s, 1H), 7.63 (d, J = 12.2 Hz, 1H), 7.30 (s, 1H), 6.83 (s, 1H), 5.33 (d, J = 12.2 Hz, 1H), 3.83 (s, 1H), 3.52 (s, 1H), 2.63 (s, 3H), 2.54 (s, 3H), 2.09 (s, 4H), 1.95 (s, 2H), 1.77 (s, 2H), 1.53 (s, 2H), 1.36 (s, 5H), 1.18 (s, 1H), 0.97-0.87 (m, 3H) ppm |
| 4 | 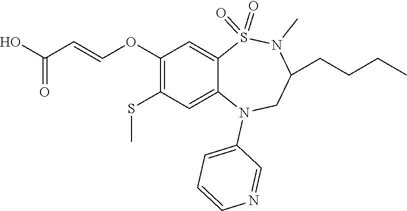 [SMILES: CCCCC1CN(c2cccnc2)c2cc(SC)c(O/C=C/C(=O)O)cc2S(=O)(=O)N1C] A mixture of two enantiomers | 478 [M + 1]⁺ | ¹H NMR (300 MHz, DMSO-d6): δ 8.08 (d, J = 5.7 Hz, 2H), 7.83 (d, J = 12.3 Hz, 1H), 7.53 (s, 1H), 7.44 (dd, J = 8.7, 4.8 Hz, 1H), 7.36-7.31 (m, 2H), 5.58 (d, J = 12.3 Hz, 1H), 4.14 (d, J = 16.2 Hz, 1H), 3.93-3.91 (m, 1H), 3.39-3.31 (m, 1H), 2.47 (s, 6H), 1.71-1.69 (m, 1H), 1.49-1.44 (m, 1H), 1.35 (s, 4H), 0.94-0.92 (m, 3H) ppm |
| 5 | 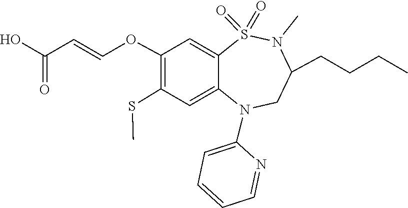 [SMILES: CCCCC1CN(c2ccccn2)c2cc(SC)c(O/C=C/C(=O)O)cc2S(=O)(=O)N1C] A mixture of two enantiomers | 478 [M + 1]⁺ | ¹H NMR (300 MHz, DMSO-d6): δ 8.15 (dd, J = 5.1, 1.8 Hz, 1H), 7.83 (d, J = 12.0 Hz, 1H), 7.52 (s, 1H), 7.49- 7.44 (m, 1H), 7.39 (s, 1H), 6.72 (dd, J = 7.2, 5.1 Hz, 1H), 6.30 (d, J = 8.7 Hz, 1H), 5.56 (d, J = 12.0 Hz, 1H), 4.66 (d, J = 13.5 Hz, 1H), 4.06-3.96 (m, 1H), 3.04 (s, 1H), 2.47 (s, 6H), 1.52 (s, 2H), 1.41-1.32 (m, 4H), 0.94-0.89 (m, 3H) ppm |
| 6 | 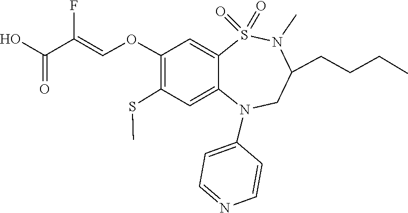 [SMILES: CCCCC1CN(c2ccncc2)c2cc(SC)c(O/C=C(\F)C(=O)O)cc2S(=O)(=O)N1C] A mixture of two enantiomers | 496 [M + 1]⁺ | ¹H NMR (300 MHz, DMSO-d6): δ 8.20 (s, 2H), 7.61 (d, J = 18 Hz, 1H), 7.53 (d, J = 15 Hz, 1H), 7.34 (s, 1H), 6.61 (s, 2H), 4.09 (d, J = 15.9 Hz, 1H), 4.00 (s, 1H), 3.33-3.08 (m, 1H), 2.50-2.47 (m, 6H), 1.92-1.72 (m, 1H), 1.50-1.21 (m, 5H), 0.95-0.82 (m, 3H) ppm |
| 7 | 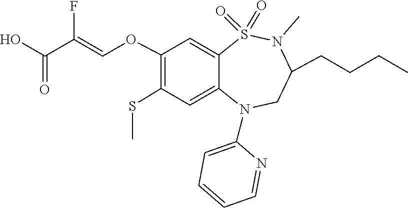 [SMILES: CCCCC1CN(c2ccccn2)c2cc(SC)c(O/C=C(\F)C(=O)O)cc2S(=O)(=O)N1C] A mixture of two enantiomers | 496 [M + 1]⁺ | ¹H NMR (300 MHz, DMSO-d6): δ 8.19-8.11 (m, 1H), 7.59 (d, J = 18 Hz, 1H), 7.47 (d, J = 1.8 Hz, 1H), 7.44 (t, J = 6.9 Hz, 1H), 7.34 (s, 1H), 6.70 (dd, J = 7.2, 5.1 Hz, 1H), 6.24 (d, J = 8.4 Hz, 1H), 4.66 (d, J = 15.0 Hz, 1H), 3.99-3.97 (m, 1H), 3.02-3.02 (m, 1H), 2.46 (s, 6H), 1.60-1.52 (m, 2H), 1.41-1.27 (m,4H), 0.94-0.85 (m, 3H) ppm |
| 8 | 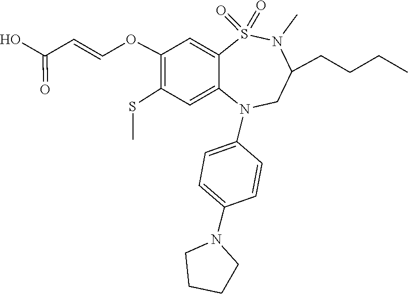 [SMILES: CCCCC1CN(c2ccc(N3CCCC3)cc2)c2cc(SC)c(O/C=C/C(=O)O)cc2S(=O)(=O)N1C] A mixture of two enantiomers | 546 [M + 1]⁺ | ¹H NMR (300 MHz, DMSO-d6): δ 7.61 (d, J = 12.3 Hz, 1H), 7.30 (s, 1H), 6.86 (s, 1H), 5.34 (d, J = 12.3 Hz, 1H), 3.93 (d, J = 11.4 Hz, 2H), 3.82-3.64 (m, 1H), 3.50-3.42 (m, 5H), 2.63 (s, 3H), 2.51 (s, 3H), 1.84 (d, J = 9.3 Hz, 4H), 1.62-1.48 (m, 2H), 1.44-1.24 (m, 4H), 0.90 (t, J = 6.6 Hz, 3H) ppm |
| 9 | 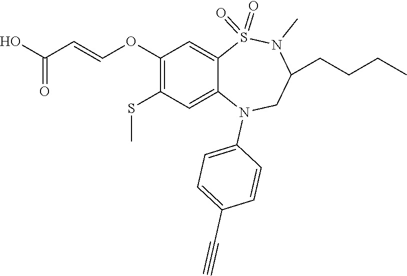 [SMILES: C#Cc1ccc(N2CC(CCCC)N(C)S(=O)(=O)c3cc(O/C=C/C(=O)O)c(SC)cc32)cc1] A mixture of two enantiomers | 502 [M + 1]⁺ | ¹H NMR (300 MHz, DMSO-d6): δ 7.69 (d, J = 12.9 Hz, 1H), 7.47 (s, 1H), 7.27 (d, J = 10.5 Hz, 3H), 6.60 (d, J = 8.4 Hz, 2H), 5.56 (d, J = 12.0 Hz, 1H), 4.05 (d, J = 15.6 Hz, 1H), 3.92-3.90 (m, 1H), 3.88-3.86 (m, 1H), 3.63-3.58 (m, 1H), 2.47 (s, 3H), 2.43 (s, 3H), 1.64-1.63 (m, 1H), 1.52-1.48 (m, 1H), 1.37-1.32 (m, 4H), 0.92 (d, J = 6.3 Hz, 3H) ppm |
| 10 | 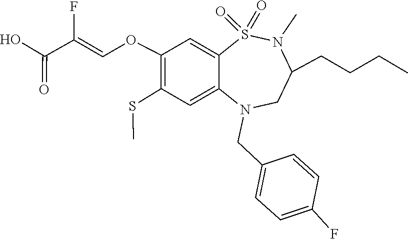 [SMILES: CCCCC1CN(Cc2ccc(F)cc2)c2cc(SC)c(O/C=C(\F)C(=O)O)cc2S(=O)(=O)N1C] A mixture of two enantiomers | 527 [M + 1]⁺ | 1H NMR (400 MHz, CDCl₃): δ 7.43 (s, 1H), 7.38-7.35 (m, 2H), 7.20 (d, J = 17.2 Hz, 1H), 7.00 (t, J = 8.8 Hz, 2H), 6.74 (s, 1H), 4.50 (d, J = 13.6 Hz, 1H), 4.30 (d, J = 13.6 Hz, 1H), 4.31 (q, J = 7.2 Hz, 2H), 3.62 (brs, 1H), 3.02 (brs, 1H), 2.87-2.83 (m, 1H), 2.52 (s, 3H), 2.30 (s, 3H), 1.41- 1.38 (m, 1H), 1.20-1.02 (m, 5H), 0.78 (t, J = 7.2 Hz, 3H) ppm |
| 11 | 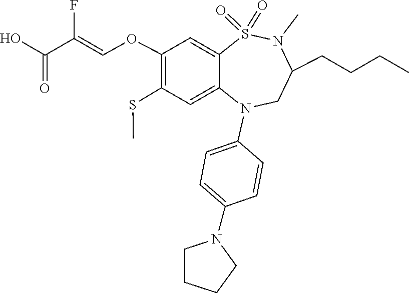 [SMILES: CCCCC1CN(c2ccc(N3CCCC3)cc2)c2cc(SC)c(O/C=C(\F)C(=O)O)cc2S(=O)(=O)N1C] A mixture of two enantiomers | 564 [M + 1]⁺ | ¹H NMR (300 MHz, DMSO-d6): δ 7.26 (s, 1H), 7.10-6.90 (m, 1H), 6.84 (d, J = 8.7 Hz, 2H), 6.73 (s, 1H), 6.49 (d, J = 8.7 Hz, 2H), 3.88 (d, J = 13.2 Hz, 1H), 3.66-3.62 (m, 1H), 3.54- 3.48 (m, 1H), 3.20-3.16 (m, 4H), 2.62 (s, 3H), 2.22 (s, 3H), 1.95-1.93 (m, 4H), 1.54-1.47 (m, 2H), 1.40- 1.24 (m, 4H), 0.90 (t, J = 6.6 Hz, 3H) ppm |
| 12 | 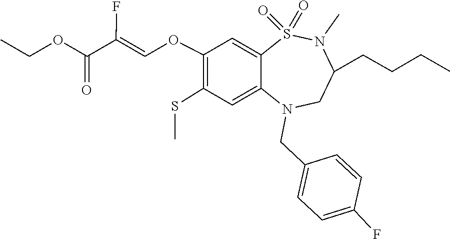 [SMILES: CCCCC1CN(Cc2ccc(F)cc2)c2cc(SC)c(O/C=C(\F)C(=O)OCC)cc2S(=O)(=O)N1C] A mixture of two enantiomers | 555 [M + 1]⁺ | ¹H NMR (400 MHz, CDCl₃): δ 7.56 (s, 1H), 7.40-7.37 (m, 1H), 7.20 (d, J = 17.2 Hz, 1H), 7.09-7.04 (m, 2H), 6.78 (s, 1H), 4.60 (d, J = 14.4 Hz, 1H), 4.40 (d, J = 14.4 Hz, 1H), 4.31 (q, J = 7.2 Hz, 2H), 3.72 (brs, 1H), 3.30 (brs, 1H), 3.00 (dd, J = 14.8, 2.4 Hz, 1H), 2.63 (s, 3H), 2.34 (s, 3H), 1.55-1.49 (m, 2H), 1.35 (t, J = 7.2 Hz, 3H), 1.29-1.11 (m, 4H), 0.85 (t, J = 6.8 Hz, 3H) ppm |
| 13 | 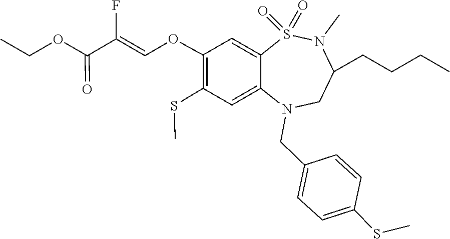 [SMILES: CCCCC1CN(Cc2ccc(SC)cc2)c2cc(SC)c(O/C=C(\F)C(=O)OCC)cc2S(=O)(=O)N1C] A mixture of two enantiomers | 584 [M + 1]⁺ | ¹H NMR (400 MHz, CDCl₃): δ 7.55 (s, 1H), 7.32-7.24 (m, 4H), 7.20 (d, J = 17.2 Hz, 1H), 6.75 (s, 1H), 4.60 (d, J = 14.8 Hz, 1H), 4.38 (d, J = 14.4 Hz, 1H), 4.31 (q, J = 7.2 Hz, 2H), 3.68 (brs, 1H), 3.30 (brs, 1H), 3.04 (d, J = 13.2 Hz, 1H), 2.66 (s, 3H), 2.49 (s, 3H), 2.33 (s, 3H), 1.64-1.57 (m, 2H), 1.35 (t, J = 7.2 Hz, 3H), 1.25- 1.19 (m, 4H), 0.85 (t, J = 7.2 Hz, 3H) ppm |
| 14 | 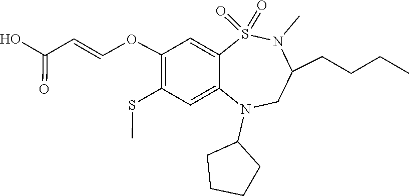 [SMILES: CCCCC1CN(C2CCCC2)c2cc(SC)c(O/C=C/C(=O)O)cc2S(=O)(=O)N1C] A mixture of two enantiomers | 469 [M + 1]⁺ | ¹H NMR (300 MHz, DMSO-d6): δ 7.61 (d, J = 12.3 Hz, 1H), 7.30 (s, 1H), 6.88 (s, 1H), 5.34 (d, J = 12.3 Hz, 1H), 4.08-4.03 (m, 1H), 3.69- 3.57 (m, 1H), 3.18-3.03 (m, 2H), 2.68 (s, 3H), 2.58 (s, 3H), 2.09-1.92 (m, 2H), 1.69-1.25 (m, 12H), 0.92 (t, J = 6.9 Hz, 3H) ppm |
| 15 | 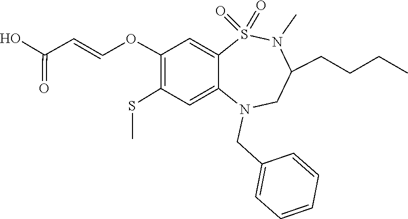 [SMILES: CCCCC1CN(Cc2ccccc2)c2cc(SC)c(O/C=C/C(=O)O)cc2S(=O)(=O)N1C] A mixture of two enantiomers | 491 [M + 1]⁺ | ¹H NMR (300 MHz, DMSO-d6): δ 7.61 (d, J = 12.0 Hz, 1H), 7.45-7.31 (m, 4H), 7.30 (d, J = 11.7 Hz, 2H), 6.82 (s, 1H), 5.34 (d, J = 12.3 Hz, 1H), 4.78 (d, J = 15.6 Hz, 1H), 4.54 (d, J = 15.6 Hz, 1H), 3.65-3.62 (m, 1H), 3.44-3.40 (m, 1H), 3.12 (d, J = 12.9 Hz, 1H), 2.60 (s, 3H), 2.33 (s, 3H), 1.52-1.46 (m, 1H), 1.25-1.09 (m, 5H), 0.82 (t, J = 6.6 Hz, 3H) ppm |
| 16 | 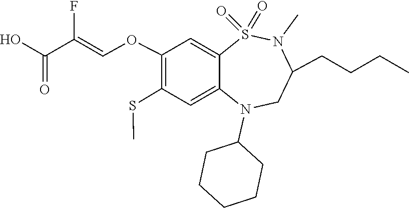 [SMILES: CCCCC1CN(C2CCCCC2)c2cc(SC)c(O/C=C(\F)C(=O)O)cc2S(=O)(=O)N1C] A mixture of two enantiomers | 501 [M + 1]⁺ | ¹H NMR (300 MHz, DMSO-d6): δ 7.15 (d, J = 7.8 Hz, 1H), 7.36 (d, J = 16.8 Hz, 1H), 6.75 (s, 1H), 3.63- 3.51 (m, 4H), 2.65 (s, 3H), 2.50 (s, 3H), 1.89 (d, J = 9.9 Hz, 2H), 1.79 (d, J = 9.9 Hz, 2H), 1.62-1.12 (m, 12H), 0.92 (t, J = 6.9 Hz, 3H) ppm |
| 17 | 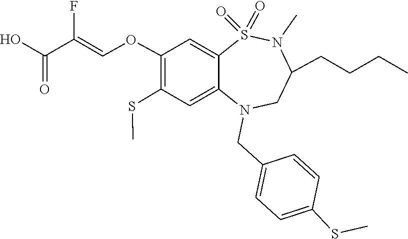 [SMILES: CCCCC1CN(Cc2ccc(SC)cc2)c2cc(SC)c(O/C=C(\F)C(=O)O)cc2S(=O)(=O)N1C] A mixture of two enantiomers | 555 [M + 1]⁺ | ¹H NMR (400 MHz, CD₃OD): δ 7.44 (s, 1H), 7.38 (d, J = 8.4 Hz, 2H), 7.25 (d, J = 8.0 Hz, 1H), 7.10 (d, J = 17.6 Hz, 1H), 6.88 (s, 1H), 4.62 (d, J = 14.8 Hz, 1H), 4.40 (d, J = 14.4 Hz, 1H), 3.69-3.65 (m, 1H), 3.21-3.18 (m, 1H), 3.06 (dd, J = 7.2, 2.0 Hz, 1H), 2.59 (s, 3H), 2.46 (s, 3H), 2.37 (s, 3H), 1.51-1.48 (m, 1H), 1.28- 1.12 (m, 5H), 0.85 (t, J = 7.2 Hz, 3H) ppm |
| 18 | 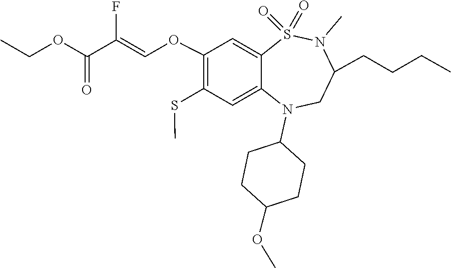 [SMILES: CCCCC1CN(C2CCC(OC)CC2)c2cc(SC)c(O/C=C(\F)C(=O)OCC)cc2S(=O)(=O)N1C] A mixture of four diastereomers | 559 [M + 1]⁺ | ¹H NMR (400 MHz, CD₃OD): δ 7.41 (s, 1H), 7.30 (d, J = 17.6 Hz, 1H), 6.88 (brs, 1H), 4.28 (q, J = 7.2 Hz, 2H), 3.56-3.46 (m, 3H), 3.37 (s, 3H), 2.72-2.67 (m, 2H), 2.50 (s, 3H), 2.12-2.07 (m, 2H), 1.93-1.37 (m, 13H), 1.35-1.26 (m, 5H), 1.29- 1.11 (m, 4H), 0.97 (t, J = 7.2 Hz, 3H) ppm |
| 19 | 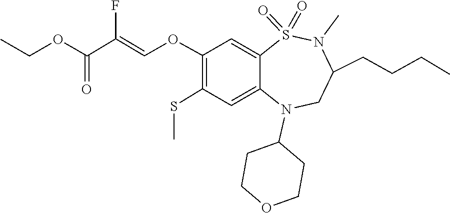 [SMILES: CCCCC1CN(C2CCOCC2)c2cc(SC)c(O/C=C(\F)C(=O)OCC)cc2S(=O)(=O)N1C] A mixture of two enantiomers | 531 [M + 1]⁺ | ¹H NMR (400 MHz, CDCl₃): δ 7.56 (s, 1H), 7.22 (d, J = 17.2 Hz, 1H), 6.94 (s, 1H), 4.32 (q, J = 7.2 Hz, 2H), 4.13-4.07 (m, 2H), 3.65 (brs, 1H), 3.51-3.42 (m, 2H), 3.27 (d, J = 14.8, 1H), 2.62 (s, 3H), 2.47 (s, 3H), 1.98- 1.90 (m, 2H), 1.79-1.76 (m, 2H), 1.57-1.24 (m, 11H), 0.95 (t, J = 7.2 Hz, 3H) ppm |
| 20 | 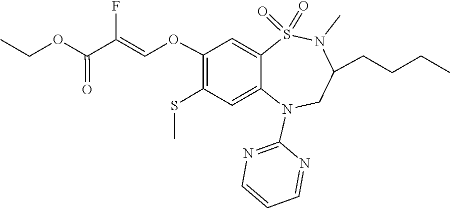 [SMILES: CCCCC1CN(c2ncccn2)c2cc(SC)c(O/C=C(\F)C(=O)OCC)cc2S(=O)(=O)N1C] A mixture of two enantiomers | 525 [M + 1]⁺ | ¹H NMR (400 MHz, CDCl₃): δ 8.36 (s, 2H), 7.61 (s, 1H), 7.34 (d, J = 16.8 Hz, 1H), 7.19 (s, 1H), 6.69 (t, J = 4.4 Hz, 1H), 4.68-4.65 (m, 1H), 4.36- 4.30 (m, 3H), 3.02-2.96 (m, 1H), 2.52 (s, 3H), 2.45 (s, 3H), 1.64-1.62 (m, 1H), 1.48-1.34 (m, 8H), 0.95 (t, J = 7.2 Hz, 3H) ppm |
| 21 | 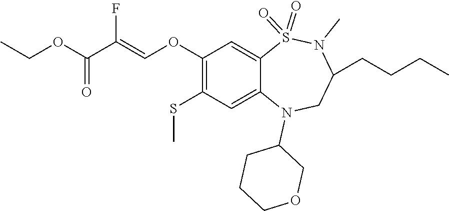 [SMILES: CCCCC1CN(C2CCCOC2)c2cc(SC)c(O/C=C(\F)C(=O)OCC)cc2S(=O)(=O)N1C] A mixture of four diastereomers | 531 [M + 1]⁺ | ¹H NMR (400 MHz, CDCl₃): δ 7.51 (s, 1H), 7.20 (d, J = 17.6Hz, 1H), 6.88 (brs, 1H), 4.31 (q, J = 8.4 Hz, 1H), 4.10 (d, J = 9.2 Hz, 1H), 3.91 (d, J = 10.8 Hz, 1H), 3.59-3.22 (m, 4H), 2.70-2.58 (m, 3H), 2.47 (s, 3H), 2.23 (d, J = 8.0 Hz, 1H), 1.79-1.36 (m, , 14H), 1.33 (t, J = 7.2 Hz, 3H), 0.96 (t, J = 6.8 Hz, 3H) ppm |
| 22 | 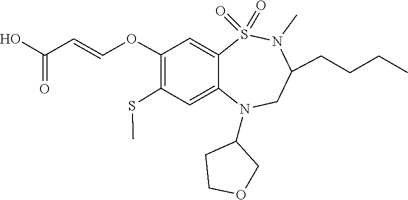 [SMILES: CCCCC1CN(C2CCOC2)c2cc(SC)c(O/C=C/C(=O)O)cc2S(=O)(=O)N1C] A mixture of four diastereomers | 471 [M + 1]⁺ | ¹H NMR (300 MHz, DMSO-d6): δ 7.62 (d, J = 12.3 Hz, 1H), 7.33 (s, 1H), 6.79 (s, 1H), 5.36 (d, J = 12.3 Hz, 1H), 4.42-4.38 (m, 1H), 4.03- 3.90 (m, 2H), 3.80-3.55 (m, 3H), 3.18 (d, J = 15.0 Hz, 1H), 2.99-2.90 (m, 1H), 2.51 (s, 3H), 2.41 (s, 3H), 2.28-2.22 (m, 1H), 1.84-1.82 (m, 1H), 1.60-1.24 (m, 6H), 0.92 (t, J = 6.6 Hz, 3H) ppm |
| 23 | 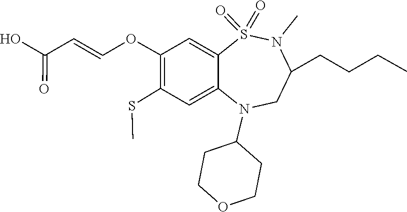 [SMILES: CCCCC1CN(C2CCOCC2)c2cc(SC)c(O/C=C/C(=O)O)cc2S(=O)(=O)N1C] A mixture of two enantiomers | 485 [M + 1]⁺ | ¹H NMR (300 MHz, DMSO-d6): δ 7.61 (d, J = 12.3 Hz, 1H), 7.30 (s, 1H), 6.86 (s, 1H), 5.34 (d, J = 12.3 Hz, 1H), 3.93 (d, J = 11.4 Hz, 2H), 3.82-3.64 (m, 1H), 3.50-3.42 (m, 5H), 2.63 (s, 3H), 2.51 (s, 3H), 1.84 (d, J = 9.3 Hz, 4H), 1.62-1.48 (m, 2H), 1.44-1.24 (m,4H), 0.90 (t, J = 6.6 Hz, 3H) ppm |
| 24 | 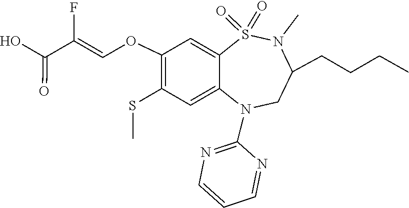 [SMILES: CCCCC1CN(c2ncccn2)c2cc(SC)c(O/C=C(\F)C(=O)O)cc2S(=O)(=O)N1C] A mixture of two enantiomers | 497 [M + 1]⁺ | ¹H NMR (400 MHz, CD₃OD): δ 8.35- 8.33 (m, 2H), 7.57 (s, 1H), 7.37 (d, J = 8.6 Hz, 1H), 7.31 (s, 1H), 6.76 (t, J = 4.8 Hz, 1H), 4.58 (d, J = 7.6 Hz, 1H), 4.26-4.22 (m, 1H), 3.19-3.13 (m, 1H), 2.51 (s, 3H), 2.46 (s, 3H), 1.62-1.57 (m, 2H), 1.54-1.40 (m, 4H), 0.98 (t, J = 7.2 Hz, 3H) ppm |
| 25 | 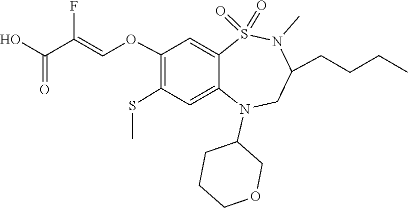 [SMILES: CCCCC1CN(C2CCCOC2)c2cc(SC)c(O/C=C(\F)C(=O)O)cc2S(=O)(=O)N1C] A mixture of four diastercomers | 503 [M + 1]⁺ | ¹H NMR (400 MHz, CD₃OD): δ 7.41 (d, J = 4.4 Hz, 1H), 7.27 (dd, J = 17.6, 7.2 Hz, 1H), 6.97 (d, J = 9.2 Hz, 1H), 4.08-4.03 (m, 1H), 3.86 (t, J = 8.4 Hz, 1H), 3.57-3.35 (m, 6H), 2.72- 2.58 (m, 3H), 2.50 (s, 3H), 2.18 (m, 1H), 1.82-1.29 (m, 19H), 0.98 (t, J = 6.2 Hz, 3H) ppm |
| 26 | 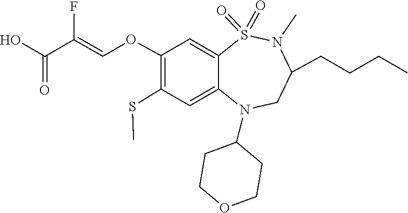 [SMILES: CCCCC1CN(C2CCOCC2)c2cc(SC)c(O/C=C(\F)C(=O)O)cc2S(=O)(=O)N1C] A mixture of two enantiomers | 503 [M + 1]⁺ | ¹H NMR (400 MHz, CDCl₃): δ 7.42 (s, 1H), 7.21 (d, J = 17.6 Hz, 1H), 6.95 (s, 1H), 4.05-4.0 (m, 2H), 3.72- 3.47 (m, 4H), 3.36-3.33 (m, 2H), 2.67 (s, 3H), 2.50 (s, 3H), 1.75-1.38 (m, 10H), 0.95 (t, J = 7.2 Hz, 3H) ppm |
| 27 | 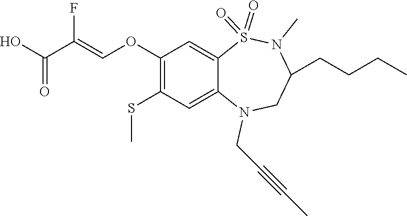 [SMILES: CC#CCN1CC(CCCC)N(C)S(=O)(=O)c2cc(O/C=C(\F)C(=O)O)c(SC)cc21] A mixture of two enantiomers | 471 [M + 1]⁺ | ¹H NMR (300 MHz, DMSO-d6): δ 7.39 (d, J = 22 Hz, 2H), 7.12 (s, 1H), 4.21-4.10 (m, 2H),3.80-3.70 (m, 1H), 3.18-2.72 (m, 4H), 2.60 (s, 3H), 1.85 (s, 3H), 1.57-1.25 (m, 7H), 0.93 (d, J = 6.4 Hz, 3H) ppm |
| 27a | 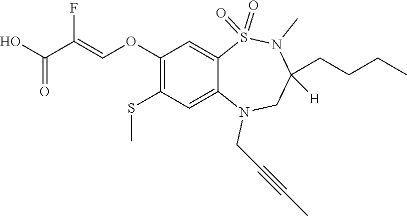 [SMILES: CC#CCN1CC(CCCC)N(C)S(=O)(=O)c2cc(O/C=C(\F)C(=O)O)c(SC)cc21] Single enantiomer I | 471 [M + 1]⁺ | ¹H NMR (300 MHz, CD₃OD): δ 7.42 (s, 1H), 7.29 (d, J = 17.7 Hz, 1H), 7.20 (s, 1H), 4.19-3.97 (m, 2H), 3.78 (s, 1H), 3.43 (t, J = 12.9 Hz, 1H), 3.36-3.24 (m, 1H), 2.70 (s, 3H), 2.52 (s, 3H), 1.85 (s, 3H), 1.74-1.62 (m, 1H), 1.58-1.29 (m, 5H), 0.90 (t, J = 6.9 Hz, 3H) ppm |
| 27b | 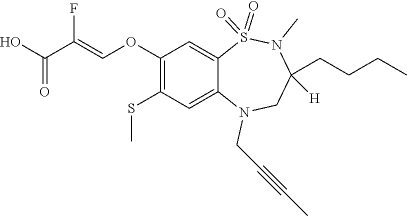 [SMILES: CC#CCN1CC(CCCC)N(C)S(=O)(=O)c2cc(O/C=C(\F)C(=O)O)c(SC)cc21] Single enantiomer II | 471 [M + 1]⁺ | ¹H NMR (300 MHz, CD₃OD): δ 7.42 (s, 1H), 7.29 (d, J = 17.7 Hz, 1H), 7.20 (s, 1H), 4.19-3.97 (m, 2H), 3.78 (s, 1H), 3.51-3.37 (m, 1H), 3.36-3.24 (m, 1H), 2.70 (s, 3H), 2.53 (s, 3H), 1.85 (s, 3H), 1.74-1.62 (m, 1H), 1.54-1.37 (m, 5H), 0.97 (t, J = 6.9 Hz, 3H) ppm |
| 28 | 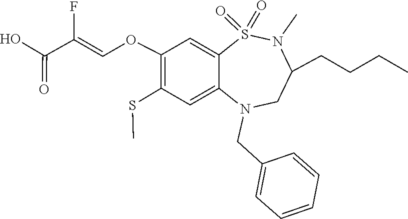 [SMILES: CCCCC1CN(Cc2ccccc2)c2cc(SC)c(O/C=C(\F)C(=O)O)cc2S(=O)(=O)N1C] A mixture of two enantiomers | 509 [M + 1]⁺ | ¹H NMR (400 MHz, CD₃OD): δ 7.44- 7.42 (m, 3H), 7.35 (t, J = 8.0 Hz, 2H), 7.29-7.19 (m, 2H), 6.86 (s, 1H), 4.70 (d, J = 14.4 Hz, 1H), 4.48 (d, J = 14.4 Hz, 1H), 3.69 (s, 1H), 3.48 (s, 1H), 3.14-3.09 (m, 1H), 2.64 (s, 3H), 2.33 (s, 3H), 1.29-1.21 (m, 6H), 0.85 (t, J = 6.8 Hz, 3H) |
| 29 | 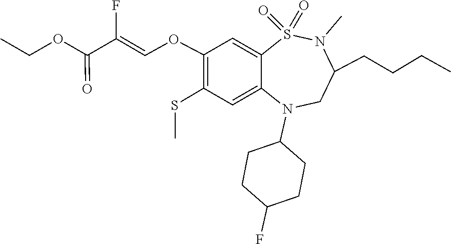 [SMILES: CCCCC1CN(C2CCC(F)CC2)c2cc(SC)c(O/C=C(\F)C(=O)OCC)cc2S(=O)(=O)N1C] A mixture of four diastereomers | 547 [M + 1]⁺ | ¹H NMR (400 MHz, CD₃OD): δ 7.41 (s, 1H), 7.30 (d, J = 17.6 Hz, 1H), 6.89 (brs, 1H), 4.86-4.62 (m, 1H), 4.28 (q, J = 7.2 Hz, 2H), 3.59 (brs, 2H), 3.38-3.33 (m, 1H), 2.72-2.67 (m, 2H), 2.50 (s, 3H), 2.18-2.12 (m, 2H), 2.03-1.38 (m, 14H), 1.31 (t, J = 7.2 Hz, 3H), 0.97 (t, J = 7.2 Hz, 3H) ppm |
| 30 | 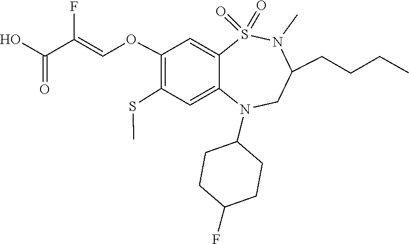 [SMILES: CCCCC1CN(C2CCC(F)CC2)c2cc(SC)c(O/C=C(\F)C(=O)O)cc2S(=O)(=O)N1C] A mixture of four diastereomers | 519 [M + 1]⁺ | ¹H NMR (400 MHz, CD₃OD): δ 7.41 (s, 1H), 7.18 (d, J = 17.6 Hz, 1H), 6.91 (brs, 1H), 4.89-4.73 (m, 1H), 3.60-3.48 (m, 2H), 3.35-3.30 (m, 3H), 2.70-2.64 (m, 2H), 2.50 (d, J = 4.4 Hz, 3H), 2.14-2.05 (m, 2H), 1.99- 1.41 (m, 12H), 0.97 (t, J = 7.2 Hz, 3H) ppm |
| 31 | 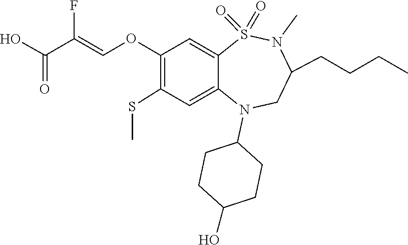 [SMILES: CCCCC1CN(C2CCC(O)CC2)c2cc(SC)c(O/C=C(\F)C(=O)O)cc2S(=O)(=O)N1C] A mixture of four diastereomers | 517 [M + 1]⁺ | ¹H NMR (300 MHz, DMSO-d6): δ 7.17 (s, 1H), 6.89 (d, J = 16.8 Hz, 1H), 6.77 (s, 1H), 3.94 (s, 1H), 3.52- 3.44 (m, 2H), 3.35 (d, J = 12.3 Hz, 1H), 2.55 (s, 3H), 2.50 (s, 3H), 2.48 (s, 1H), 1.95-1.77 (m, 4H), 1.76- 1.39 (m, 6H), 1.36-1.24 (m, 4H), 0.90 (d, J = 6.9 Hz, 3H) ppm |
| 32 | 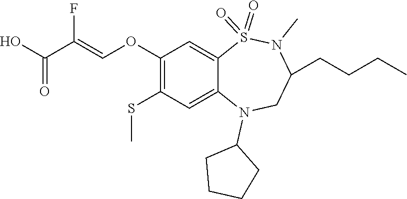 [SMILES: CCCCC1CN(C2CCCC2)c2cc(SC)c(O/C=C(\F)C(=O)O)cc2S(=O)(=O)N1C] A mixture of two enantiomers | 487 [M + 1]⁺ | ¹H NMR (400 MHz, DMSO-d6): δ 7.39-7.34 (m, 2H), 6.86 (s, 1H), 4.02 (s, 1H), 3.62 (s, 1H), 3.48-3.36 (m, 1H), 3.16 (s, 1H), 2.60-2.56 (m, 3H), 2.50-2.43 (m, 3H), 2.04-2.02 (m, 1H), 1.91 (s, 1H), 1.69-1.32 (m, 12H), 0.92 (t, J = 6.4 Hz, 3H) ppm |
| 32a | 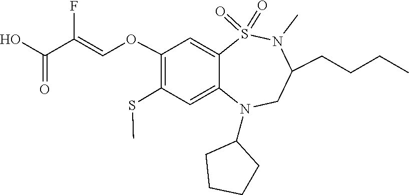 [SMILES: CCCCC1CN(C2CCCC2)c2cc(SC)c(O/C=C(\F)C(=O)O)cc2S(=O)(=O)N1C] Single enantiomer I | 487 [M + 1]⁺ | ¹H NMR (300 MHz, CD₃OD): δ 7.42 (s, 1H), 7.31 (d, J = 17.7 Hz, 1H), 6.96 (s, 1H), 4.07-3.99 (m, 1H), 3.71-3.54 (m, 1H), 3.21-3.07 (m, 2H), 2.74-2.62 (m, 3H), 2.51 (s, 3H), 2.07-1.98 (m, 2H), 1.76-1.48 (m, 12H), 0.99 (t, J = 6.9 Hz, 3H) ppm |
| 32b | 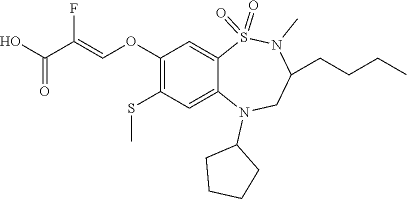 [SMILES: CCCCC1CN(C2CCCC2)c2cc(SC)c(O/C=C(\F)C(=O)O)cc2S(=O)(=O)N1C] | 487 [M + 1]⁺ | ¹H NMR (300 MHz, CD₃OD): δ 7.42 (s, 1H), 7.28 (d, J = 17.7 Hz, 1H), 6.95 (s, 1H), 4.05-4.00 (m, 1H), 3.71-3.53 (m, 1H), 3.26-3.21 (m, 2H), 2.89-2.63 (m, 3H), 2.50 (s, 3H), 2.10-2.02 (m, 2H), 1.76-1.44 (m, 12H), 0.96 (t, J = 6.9 Hz, 3H) ppm |
| 33 | 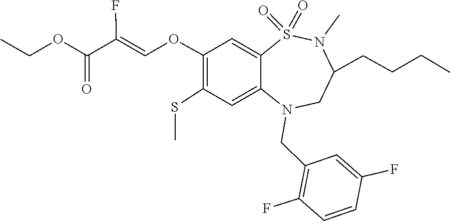 [SMILES: CCCCC1CN(Cc2cc(F)ccc2F)c2cc(SC)c(O/C=C(\F)C(=O)OCC)cc2S(=O)(=O)N1C] A mixture of two enantiomers | 573 [M + 1]⁺ | ¹H NMR (400 MHz, CD₃OD): δ 7.47 (s, 1H), 7.37 (s, 1H), 7.32 (s, 1H), 7.31-7.27 (m, 1H), 7.17-7.12 (m, 1H), 7.06-7.01 (m, 1H), 6.87 (s, 1H), 4.78 (d, J = 15.6 Hz, 1H), 4.56 (d, J = 15.6 Hz, 1H), 3.82-3.74 (m, 2H), 3.40-3.35 (m, 1H), 3.14-3.11 (m, 1H), 2.64 (s, 3H), 2.38 (s, 3H), 1.41-1.24 (m, 9H), 0.93-0.87 (m, 3H) ppm |
| 34 | 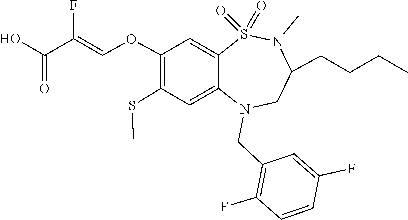 [SMILES: CCCCC1CN(Cc2cc(F)ccc2F)c2cc(SC)c(O/C=C(\F)C(=O)O)cc2S(=O)(=O)N1C] A mixture of two enantiomers | 545 [M + 1]⁺ | ¹H NMR (400 MHz, CD₃OD): δ 7.46 (s, 1H), 7.34-7.29 (m, 1H), 7.22 (d, J = 18.0 Hz, 1H), 7.17-7.11 (m, 1H), 7.06-7.01 (m, 1H), 6.87 (s, 1H), 4.75 (d, J = 14.8 Hz, 1H), 4.55 (d, J = 15.6 Hz, 1H), 3.84-3.74 (m, 1H), 3.40-3.35 (m, 1H), 3.14-3.05 (m, 1H), 2.61 (s, 3H), 2.38 (s, 3H), 1.40- 1.22 (m, 6H), 0.93-0.87 (m, 3H) ppm |
| 35 | 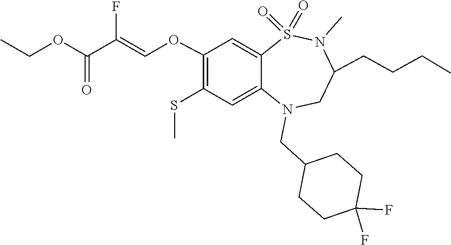 [SMILES: CCCCC1CN(CC2CCC(F)(F)CC2)c2cc(SC)c(O/C=C(\F)C(=O)OCC)cc2S(=O)(=O)N1C] A mixture of two enantiomers | 579 [M + 1]⁺ | ¹H NMR (400 MHz, CD₃OD): δ 7.47 (s, 1H), 7.33 (d, J = 17.6 Hz, 1H), 6.98 (s, 1H), 4.31-4.26 (m, 2H), 3.88-3.80 (m, 1H), 3.48-3.34 (m, 1H), 3.18-3.03 (m, 3H), 2.57 (s, 3H), 2.52 (s, 3H), 2.13-1.90 (m, 4H), 1.82-1.57 (m, 3H), 1.54-1.36 (m, 6H), 1.32 (t, J = 7.2 Hz, 3H), 1.28- 1.12 (m, 2H), 1.00-0.94 (m, 3H) ppm |
| 36 | 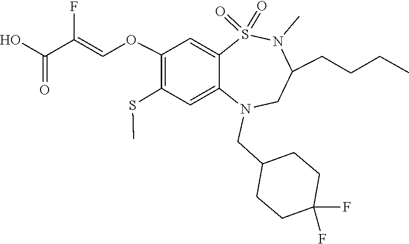 [SMILES: CCCCC1CN(CC2CCC(F)(F)CC2)c2cc(SC)c(O/C=C(\F)C(=O)O)cc2S(=O)(=O)N1C] A mixture of two enantiomers | 551 [M + 1]⁺ | ¹H NMR (400 MHz, CD₃OD): δ 7.45 (s, 1H), 7.21 (d, J = 20.4 Hz, 1H), 6.98 (s, 1H), 3.91-3.79 (m, 1H), 3.44-3.35 (m, 1H), 3.16-2.99 (m, 3H), 2.54 (s, 3H), 2.50 (s, 3H), 2.15- 1.90 (m, 4H), 1.82-1.61 (m, 3H), 1.54-1.34 (m, 6H), 1.34-1.11 (m, 2H), 1.00-0.94 (m, 3H) ppm |
| 37 | 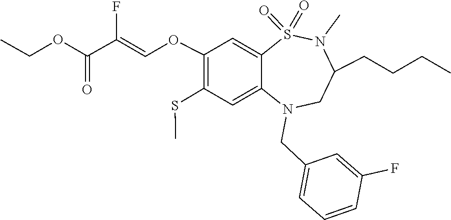 [SMILES: CCCCC1CN(Cc2cccc(F)c2)c2cc(SC)c(O/C=C(\F)C(=O)OCC)cc2S(=O)(=O)N1C] A mixture of two enantiomers | 555 [M + 1]⁺ | ¹H NMR (400 MHz, CDCl₃): δ 7.55 (s, 1H), 7.38-7.32 (m, 1H), 7.24 (s, 1H), 7.20-7.18 (m, 1H), 7.11 (d, J = 9.6 Hz, 1H), 7.03-6.98 (m, 1H), 6.75 (s, 1H), 4.64 (d, J = 15.2 Hz, 1H), 4.45 (d, J = 14.8 Hz, 1H), 4.32 (q, J = 7.2 Hz, 2H), 3.76 (s, 1H), 3.42-3.36 (m, 1H), 3.05 (dd, J = 15.2, 2.0 Hz, 1H), 2.67 (s, 3H), 2.31 (s, 3H), 1.58- 1.53 (m, 1H), 1.35 (t, J = 7.2 Hz, 3H), 1.29-1.15 (m, 5H), 0.88-0.84 (m, 3H) ppm |
| 38 | 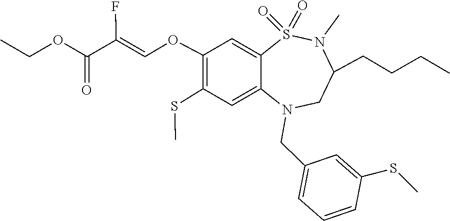 [SMILES: CCCCC1CN(Cc2cccc(SC)c2)c2cc(SC)c(O/C=C(\F)C(=O)OCC)cc2S(=O)(=O)N1C] A mixture of two enantiomers | 583 [M + 1]⁺ | ¹H NMR (400 MHz, CDCl₃): δ 7.55 (s, 1H), 7.33 (s, 1H), 7.28-7.23 (m, 2H), 7.19-7.16 (m, 1H), 7.12 (d, J = 15.2 Hz, 1H), 6.81 (s, 1H), 4.61 (d, J = 14.8 Hz, 1H), 4.43 (d, J = 14.4 Hz, 1H), 4.32 (q, J = 7.2 Hz, 2H), 3.77 (s, 1H), 3.32-3.29 (m, 1H), 3.06 (dd, J = 15.2, 2.0 Hz, 1H), 2.64 (s, 3H), 2.49 (s, 3H), 2.33 (s, 3H), 1.57-1.51 (m, 1H), 1.35 (t, J = 7.2 Hz, 3H), 1.31- 1.13 (m, 5H), 0.85 (t, J = 6.8 Hz, 3H) ppm |
| 39 | 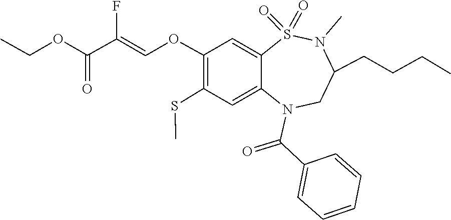 [SMILES: CCCCC1CN(C(=O)c2ccccc2)c2cc(SC)c(O/C=C(\F)C(=O)OCC)cc2S(=O)(=O)N1C] A mixture of two enantiomers | 551 [M + 1]⁺ | ¹H NMR (400 MHz, CDCl₃): δ 7.55 (s, 1H), 7.40 (d, J = 14.4 Hz, 2H), 7.30-7.26 (m, 2H), 7.23-7.19 (m, 2H), 6.47 (s, 1H), 4.88 (d, J = 14.0 Hz, 1H), 4.32 (q, J = 6.8 Hz, 3H), 2.81-2.75 (m, 1H), 2.57 (s, 2H), 2.03 (s, 3H), 1.65-1.64 (m, 1H), 1.53-1.40 (m, 5H), 1.37 (t, J = 7.2 Hz, 3H), 1.30-1.22 (m, 1H), 0.97 (t, J = 6.8 Hz, 3H) ppm |
| 40 | 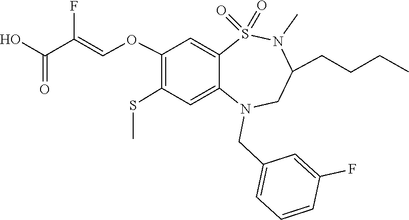 [SMILES: CCCCC1CN(Cc2cccc(F)c2)c2cc(SC)c(O/C=C(\F)C(=O)O)cc2S(=O)(=O)N1C] A mixture of two enantiomers | 527 [M + 1]⁺ | ¹H NMR (400 MHz, CD₃OD): δ 7.45 (s, 1H), 7.39-7.34 (m, 1H), 7.27- 7.21 (m, 3H), 7.02-6.98 (m, 1H), 6.86 (s, 1H), 4.72 (d, J = 7.4 Hz, 1H), 4.50 (d, J = 7.4 Hz, 1H), 3.75 (s, 1H), 3.31-3.29 (m, 1H), 3.09-3.51(m, 1H), 2.62 (s, 3H), 2.36 (s, 3H), 1.55- 1.53 (m, 1H), 1.30-1.18 (m, 5H), 0.87 (t, J = 6.8 Hz, 3H) ppm |
| 41 | 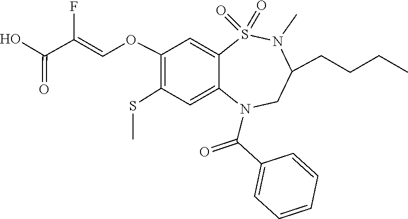 [SMILES: CCCCC1CN(C(=O)c2ccccc2)c2cc(SC)c(O/C=C(\F)C(=O)O)cc2S(=O)(=O)N1C] A mixture of two enantiomers | 523 [M + 1]⁺ | ¹H NMR (400 MHz, CD₃OD): δ 7.52 (s, 1H), 7.39 (d, J = 3.4 Hz, 2H), 7.33- 7.23 (m, 4H), 6.79 (s, 1H), 4.68 (d, J = 6.6 Hz, 1H), 4.30 (d, J = 4.4 Hz, 1H), 3.08-3.02 (m, 1H), 2.56 (s, 3H), 2.09 (s, 3H), 1.65-1.62 (m, 2H), 1.51-1.42 (m, 4H), 0.99 (t, J = 7.2 Hz, 3H) ppm |
| 42 | 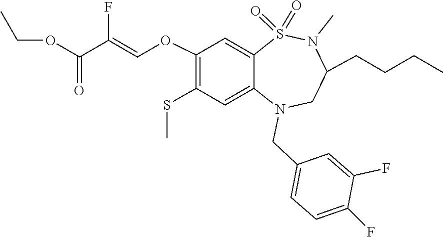 [SMILES: CCCCC1CN(Cc2ccc(F)c(F)c2)c2cc(SC)c(O/C=C(\F)C(=O)OCC)cc2S(=O)(=O)N1C] A mixture of two enantiomers | 573 [M + 1]⁺ | ¹H NMR (400 MHz, DMSO-d6): δ 7.62 (d, J = 18.8 Hz, 1H), 7.49-7.34 (m, 3H), 7.27 (s, 1H), 6.82 (m, 1H), 4.79 (d, J = 14.8 Hz, 1H), 4.53-4.46 (m, 1H), 4.26 (dd, J = 14.0 Hz, J = 6.8 Hz, 2H), 3.67 (s, 1H), 3.24 (s, 1H), 3.08 (d, J = 14.0 Hz, 1H), 2.54 (s, 3H), 2.38 (s, 3H), 1.46 (s, 1H), 1.35- 1.15 (m, 8H), 0.84 (t, J = 6.8 Hz, 3H) ppm |
| 43 | 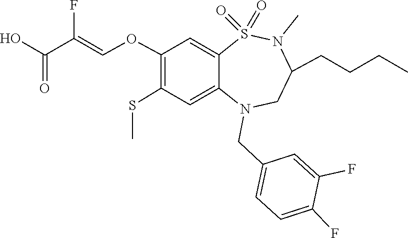 [SMILES: CCCCC1CN(Cc2ccc(F)c(F)c2)c2cc(SC)c(O/C=C(\F)C(=O)O)cc2S(=O)(=O)N1C] A mixture of two enantiomers | 545 [M + 1]⁺ | ¹H NMR (400 MHz, DMSO-d6): δ 13.52 (s, 1H), 7.54-7.40 (m, 4H), 7.27 (s, 1H), 6.83 (s, 1H), 4.78 (d, J = 14.8 Hz, 1H), 4.52 (d, J = 14.8 Hz, 1H), 3.66 (s, 1H), 3.56 (s, 1H), 3.07 (d, J = 14.0 Hz, 1H), 2.53 (s, 3H), 2.39 (s, 3H), 1.45 (s, 1H), 1.29-1.06 (m, 5H), 0.82 (t, J = 16.0 Hz, 3H) ppm |
| 44 | 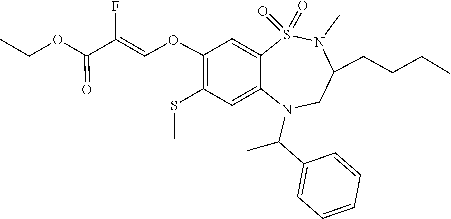 [SMILES: CCCCC1CN(C(C)c2ccccc2)c2cc(SC)c(O/C=C(\F)C(=O)OCC)cc2S(=O)(=O)N1C] A mixture of four diastereomers | 551 [M + 1]⁺ | ¹H NMR (400 MHz, CDCl₃): δ 7.47 (s, 1H), 7.34-7.30 (m, 2H), 7.25- 7.19 (m, 3H), 7.13 (t, J = 17.6 Hz, 1H), 6.58 (brs, 1H), 4.77 (d, J = 5.6 Hz, 1H), 4.24 (q, J = 7.2 Hz, 2H), 3.65 (d, J = 3.2 Hz, 1H), 3.33-3.27 (m, 1H), 2.71-2.66 (m, 3H), 2.00 (s, 3H), 1.63-1.54 (m, 5H), 1.29-1.18 (m, 8H), 0.82 (t, J = 6.8 Hz, 3H) ppm |
| 45 | 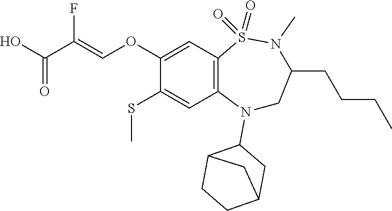 [SMILES: CCCCC1CN(C2CC3CCC2C3)c2cc(SC)c(O/C=C(\F)C(=O)O)cc2S(=O)(=O)N1C] A mixture of diastereomers | 513 [M + 1]⁺ | ¹H NMR (400 MHz, CD₃OD): δ 7.29 (d, J = 3.6 Hz, 1H), 7.20 (dd, J = 17.6, 4.8 Hz, 1H), 6.73 (s, 1H), 5.07 (d, J = 10.0 Hz, 1H), 3.67-3.64 (m, 2H), 3.22 (d, J = 10.8 Hz, 1H), 2.81 (s, 3H), 2.53 (s, 1H), 2.46 (s, 3H), 2.24 (s, 1H), 2.04 (dd, J = 14.0, 10.0 Hz, 1H), 1.79-1.69 (m, 2H), 1.63-1.58 (m, 1H), 1.49- 1.29 (m, 8H), 1.22 (s, 1H), 1.19- 1.06 (m, 1H), 0.96 (t, J = 6.8 Hz, 3H) ppm |
| 46 | 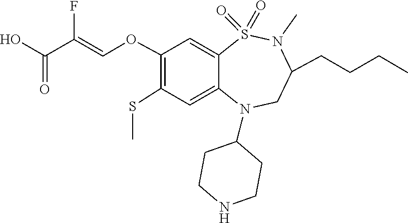 [SMILES: CCCCC1CN(C2CCNCC2)c2cc(SC)c(O/C=C(\F)C(=O)O)cc2S(=O)(=O)N1C] A mixture of two enantiomers | 502 [M + 1]⁺ | ¹H NMR (300 MHz, DMSO-d6): δ 7.23 (s, 1H), 6.95 (s, 1H), 6.89 (s, 1H), 3.90-3.80 (m, 1H), 3.60-3.50 (m, 2H), 3.34 (t, J = 10.2 Hz, 2H), 3.20 (d, J = 14.7 Hz, 1H), 3.06 (t, J = 10.8 Hz, 1H), 2.93 (t, J = 10.8 Hz, 1H), 2.53 (s, 6H), 2.09-1.70 (m, 4H), 1.52- 1.24 (m, 6H), 0.92 (d, J = 6.9 Hz, 3H) ppm |
| 47 | 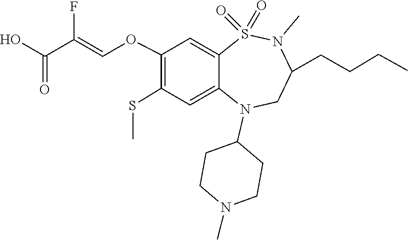 [SMILES: CCCCC1CN(C2CCN(C)CC2)c2cc(SC)c(O/C=C(\F)C(=O)O)cc2S(=O)(=O)N1C] A mixture of two enantiomers | 516 [M + 1]⁺ | ¹H NMR (300 MHz, DMSO-d6): δ 7.24 (s, 1H), 6.98 (s, 1H), 6.89 (d, J = 15.3 Hz, 1H), 3.70-3.54 (m, 2H), 3.37 (t, J = 10.5 Hz, 2H), 3.20 (t, J = 6.6 Hz, 1H), 3.03-2.84 (m, 2H), 2.75 (s, 4H), 2.54 (s, 3H), 2.40 (s, 3H), 2.12-1.83 (m, 4H), 1.48-1.34 (m, 6H), 0.93 (d, J = 6.6 Hz, 3H) ppm |
| 48 | 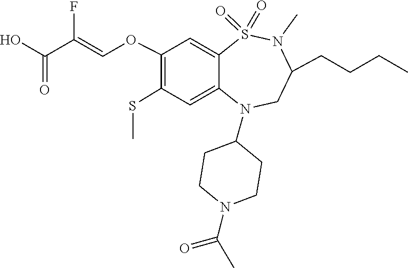 [SMILES: CCCCC1CN(C2CCN(C(C)=O)CC2)c2cc(SC)c(O/C=C(\F)C(=O)O)cc2S(=O)(=O)N1C] A mixture of two enantiomers | 544 [M + 1]⁺ | ¹H NMR (300 MHz, DMSO-d6): δ 7.37-7.31 (m, 2H), 6.84 (s, 1H), 4.49 (d, J = 12.6 Hz, 1H), 3.92-3.70 (m, 2H), 3.31-3.02 (m, 4H), 2.74- 2.60 (m, 4H), 2.50 (s, 3H), 2.04 (s, 3H), 1.86 (d, J = 11.4 Hz, 2H), 1.51- 1.33 (m, 8H), 0.92 (d, J = 6.6 Hz, 3H) ppm |
| 49 | 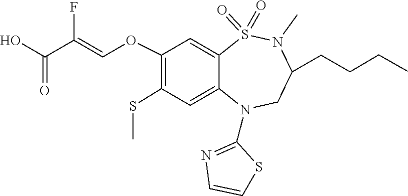 [SMILES: CCCCC1CN(c2nccs2)c2cc(SC)c(O/C=C(\F)C(=O)O)cc2S(=O)(=O)N1C] A mixture of two enantiomers | 502 [M + 1]⁺ | ¹H NMR (300 MHz, DMSO-d6): δ 7.55 (s, 1H), 7.40 (s, 1H), 7.20 (d, J = 3.6 Hz, 2H), 6.80 (d, J = 3.6 Hz, 1H), 4.30 (d, J = 15.9 Hz, 1H), 4.12 (d, J = 9.0 Hz, 1H), 3.32-3.23 (m, 1H), 2.48 (s, 3H), 2.47 (s, 3H), 1.53-1.51 (m, 2H), 1.37-1.36 (m , 4H), 0.91 (d, J = 6.6 Hz, 3H) ppm |
| 50 | 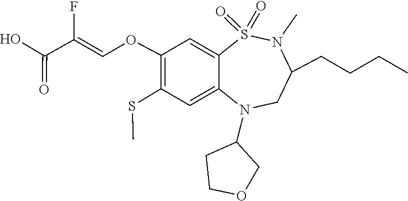 [SMILES: CCCCC1CN(C2CCOC2)c2cc(SC)c(O/C=C(\F)C(=O)O)cc2S(=O)(=O)N1C] A mixture of four diastereomers | 489 [M + 1]⁺ | ¹H NMR (300 MHz, CD₃OD): δ 7.45 (s, 1H), 7.24 (d, J = 16 Hz, 1H), 6.88 (s, 1H), 4.37-4.33 (m, 1H), 4.06- 3.96 (m, 2H), 3.90-3.83 (m, 2H), 3.72-3.63 (m, 1H), 3.22-3.13 (m, 1H), 2.98 (s, 1H), 2.56-2.51 (m, 6H), 2.36-2.27 (m, 1H), 1.92 (s, 1H), 1.59 (s, 1H), 1.48-1.42 (m, 5H), 0.97 (t, J = 6.8 Hz, 3H) ppm |
| 51 | 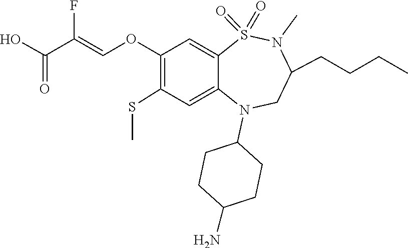 [SMILES: CCCCC1CN(C2CCC(N)CC2)c2cc(SC)c(O/C=C(\F)C(=O)O)cc2S(=O)(=O)N1C] A mixture of four diastereomers | 516 [M + 1]⁺ | ¹H NMR (400 MHz, DMSO-d6): δ 10.30 (s, 1H), 7.34-7.20 (m, 2H), 7.18 (s, 1H), 7.01 (d, J = 18.8 Hz, 1H), 6.83 (s, 1H), 3.83 (s, 1H), 3.27- 3.14 (m, 2H), 2.69-2.56 (m, 2H), 2.52 (s, 6H), 2.41-2.18 (m, 3H), 2.00-1.81 (m, 3H), 1.78-1.57 (m, 3H), 1.50-1.22 (m, 5H), 0.90 (t, J = 6.8 Hz, 3H) ppm |
| 52 | 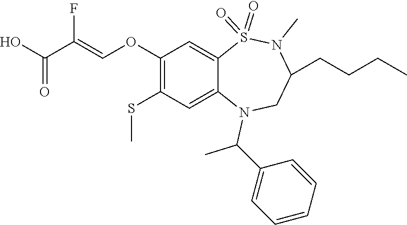 [SMILES: CCCCC1CN(C(C)c2ccccc2)c2cc(SC)c(O/C=C(\F)C(=O)O)cc2S(=O)(=O)N1C] A mixture of four diastereomers | 523 [M + 1]⁺ | ¹H NMR (400 MHz, CD₃OD): δ 7.43 (s, 1H), 7.39 (s, 2H), 7.38 (s, 2H), 7.30-7.25 (m, 1H), 7.17 (d, J = 17.6 Hz, 1H), 6.85-6.68 (m, 1H), 3.80- 3.67 (m, 1H), 3.46-3.32 (m, 1H), 3.25-3.16 (m, 1H), 2.75-2.55 (m, 3H), 2.13 (s, 3H), 1.64 (s, 3H), 1.59- 1.48 (m, 1H), 1.39-1.17 (m, 6H), 0.94-0.85 (m, 3H) ppm |
| 53 | 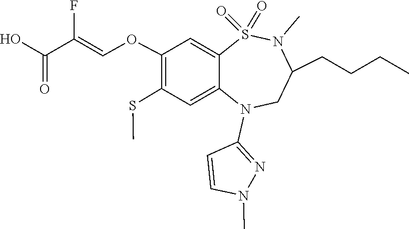 [SMILES: CCCCC1CN(c2ccn(C)n2)c2cc(SC)c(O/C=C(\F)C(=O)O)cc2S(=O)(=O)N1C] A mixture of two enantiomers | 499 [M + 1]⁺ | ¹H NMR (300 MHz, CD₃OD): δ 7.50 (s, 1H), 7.36-7.24 (m, 3H), 5.47 (s, 1H), 4.12 (d, J = 15.6 Hz, 1H), 4.09- 3.98 (m, 1H), 3.73 (s, 3H), 2.59 (s, 3H), 2.41 (s, 3H), 1.58-1.55 (m, 2H), 1.55-1.28 (m, 4H), 0.96 (t, J = 6.9 Hz, 3H) ppm |
| 54 | 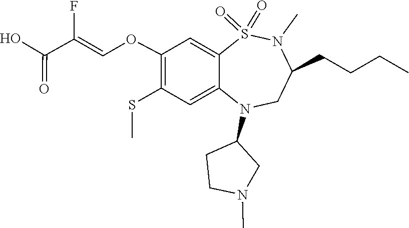 [SMILES: CCCC[C@H]1CN([C@@H]2CCN(C)C2)c2cc(SC)c(O/C=C(\F)C(=O)O)cc2S(=O)(=O)N1C] A mixture of two enantiomers with defined relative stereochemistry of the n-Butyl and N-methylpyrrolid-3-yl | 502 [M + 1]⁺ | ¹H NMR (300 MHz, CD₃OD): δ 7.47 (s, 1H), 7.17 (d, J = 18.9 Hz, 1H), 6.93 (s, 1H), 4.41-4.38 (m, 1H), 3.93-3.88 (m, 1H), 3.07-3.02 (m, 2H), 2.80 (s, 3H), 2.52 (s, 3H), 2.52 (m, 3H), 2.52-2.49 (m, 1H), 1.97- 1.93 (m, 1H), 1.52-1.28 (m, 7H), 0.99 (d, J = 6.9 Hz, 3H) ppm |
| 55 | 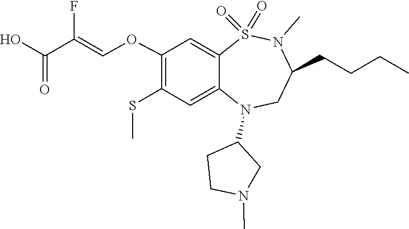 A mixture of two enantiomers with defined relative stereochemistry of the n-Butyl and N-methylpyrrolid-3-yl | 502 [M + 1]⁺ | ¹H NMR (300 MHz, CD₃OD): δ 7.47 s, 1H), 7.12 (d, J = 19.2 Hz, 1H), 7.00 (s, 1H), 4.42 (m, 1H), 3.90- 3.86 (m, 1H), 3.30-3.10 (m, 2H), 3.00-2.74 (m, 1H), 2.68 (s, 3H), 2.52 (s, 3H), 2.49-2.38 (m, 4H), 2.20-2.15 (m, 1H), 1.46-1.28 (m, 7H), 0.99 (d, J = 6.9 Hz, 3H) ppm |
| 56 | 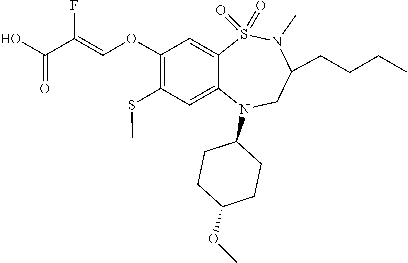 [SMILES: CCCCC1CN([C@H]2CC[C@H](OC)CC2)c2cc(SC)c(O/C=C(\F)C(=O)O)cc2S(=O)(=O)N1C] A mixture of two diastereomers with a defined stereochemistry of the 1,4-disubstitutedhexyl moiety | 531 [M + 1]⁺ | ¹H NMR (400 MHz CD₃OD): δ 7.40 (s, 1H), 7.20 (d, J = 17.6 Hz, 1H), 6.88 (brs, 1H), 3.53-3.47 (m, 3H), 3.35 (s, 3H), 3.35-3.30 (m, 3H), 2.71-2.64 (m, 2H), 2.50 (s, 3H), 2.11-2.05 (m, 2H), 1.93-1.37 (m, 12H), 0.97 (t, J = 7.2 Hz, 3H) ppm |
| 57 | 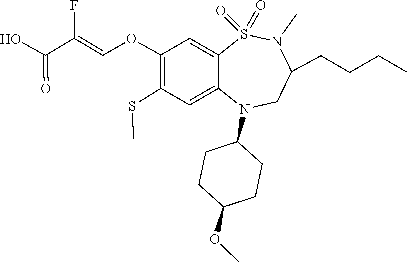 [SMILES: CCCCC1CN([C@H]2CC[C@@H](OC)CC2)c2cc(SC)c(O/C=C(\F)C(=O)O)cc2S(=O)(=O)N1C] A mixture of two diastereomers with a defined stereochemistry of the 1,4-disubstitutedhexyl moiety | 531 [M + 1]⁺ | ¹H NMR (400 MHz, CD₃OD): δ 7.40 (s, 1H), 7.20 (d, J = 17.6 Hz, 1H), 6.85 (brs, 1H), 3.53-3.47 (m, 2H), 3.35 (s, 3H), 3.35-3.30 (m, 4H), 2.74-2.67 (m, 2H), 2.50 (s, 3H), 2.21-2.14 (m, 2H), 2.09-1.96 (m, 2H), 1.73-1.31 (m, 10H), 0.97 (t, J = 7.2 Hz, 4H) ppm |
| 58 | 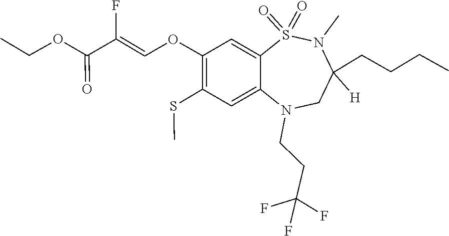 [SMILES: CCCCC1CN(CCC(F)(F)F)c2cc(SC)c(O/C=C(\F)C(=O)OCC)cc2S(=O)(=O)N1C] Single enantiomer I | 542 [M + 1]⁺ | ¹H NMR (400 MHz, CD₃OD): δ 7.43 (s, 1H), 7.35 (d, J = 18.0 Hz, 1H), 6.87 (s, 1H), 4.31-4.25 (m, 2H), 3.83-3.77 (m, 2H), 3.61-3.48 (m, 2H), 3.20 (d, J = 2.8 Hz, 1H), 2.69 (s, 3H), 2.51 (s, 3H), 1.75-1.62 (m, 2H), 1.50-1.37 (m, 6H), 1.33 (t, J = 7.2 Hz, 3H), 0.98 (t, J = 7.2 Hz, 3H) ppm |
| 59 | 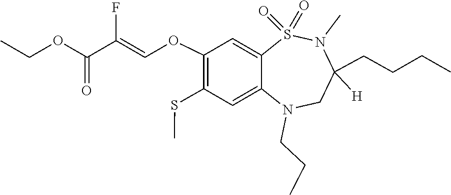 [SMILES: CCCCC1CN(CCC)c2cc(SC)c(O/C=C(\F)C(=O)OCC)cc2S(=O)(=O)N1C] Single enantiomer I | 489 [M + 1]⁺ | ¹H NMR (400 MHz, CD₃OD): δ 7.40 (s, 1H), 7.32 (d, J = 17.6 Hz, 1H), 6.82 (s, 1H), 4.30 (dd, J = 14.4, 7.2 Hz,2H), 3.72 (s, 1H), 3.51-3.40 (m, 2H), 3.17 (d, J = 2.8 Hz, 1H), 2.69 (s, 3H), 2.49 (s, 3H), 1.72-1.63 (m, 3H), 1.53-1.34 (m, 6H), 1.33 (t, J = 7.2 Hz, 3H), 1.01-0.91 (m, 6H) ppm |

### V. Biological Data

### HepG2-NTCP Infection Protocol

HepG2 cells expressing the sodium taurocholate cotransporting polypeptide (HepG2-NTCP) were maintained in culture using HepG2-NTCP growth medium (DMEM (HyClone, Cat #SH30243.02) supplemented with 10% FBS, 150 μg/mL G418 (Alfa Aesar, Cat #J62671), 50 U/mL penicillin-streptomycin (Invitrogen, Cat #15140-122), and 0.5 μg/mL blasticidin (Sigma, Cat #15205)). Prior to infection, the cells were washed twice with 1×DPBS (Invitrogen, Cat #14190-136) and treated with 3 mL of 0.05% trypsin (Invitrogen, Cat #25200-056) to dissociate the cells. Following dissociation, 10 mL of HepG2-NTCP growth medium was added to the cells to neutralize the trypsin and the cells were then centrifuged at 1,300 rpm for 5 minutes. Following centrifugation, the cells were resuspended in 10 mL of HepG2-NTCP growth medium, counted, and then centrifuged at 1,300 rpm for 5 minutes. The cell pellet was resuspended in DMEM supplemented with 5% FBS, 50 U/mL penicillin-streptomycin, 4% PEG-8000 (Hamilton Research, Cat #HR2-515), and 1% DMSO (Sigma, Cat #D4540) to a density of 5.6×10⁵ cells/mL and infected with HBV at an MOI of 50. Immediately after infection, 90 μL of the cell/HBV mixture was added to a 96-well plate containing 10 μL of compound and incubated at 37° C. for 24 hours (2% final DMSO concentration). After the incubation, the infection media was removed and replaced with DMEM supplemented with 5% FBS, 50 U/mL penicillin-streptomycin, and 1% DMSO and incubated for an additional 72 hours. At the end of the incubation, the plates were spun at 1,800 rpm for 8 minutes and the supernatant was removed for HBeAg quantification using electrochemiluminescence enzyme-linked immunosorbent assays (ECL-ELISA).

To conduct the HBeAg ECL-ELISA, Lumitrack high-binding 96-well plates (Greiner, Cat #655074) were treated with 625 ng/mL HBeAg mAb (Biocheck, Cat #70426) in 1×DPBS for 2 hours at 25° C. with shaking. The HBeAg mAb solution was then removed and the plates treated with 1×DPBS containing 0.5% bovine serum albumin (BSA) (Sigma, Cat #A7030-100 g) for 2 hours at 25° C. with shaking. The HBeAg-coated plates were then washed 4 times with 1×DPBS containing 0.05% Tween 20 (DPBS-T) (Thermo Fisher Scientific, Cat #J61544-K2). Following the wash, 90 μL of HRP-conjugated antibody (Fitzgerald, Cat #61-H10K), diluted 1:8,000 in 1×DPBS-T containing 0.5% BSA, was added to the HBeAg-coated plates along with 10 μL of sample. The plates were then incubated for 2 hours at 25° C. with shaking. Following the incubation, the sample was then removed and 200 μL of 1×PBS-T was added and the plates were incubated for 10 minutes at 25° C. with shaking. The plates were then washed 6 times with 1×PBS-T and blotted dry. 80 μL of ECL substrate (Millipore, Cat #WBKLS0500) was then added to the plate and the luminescence was measured using a Tecan M1000 Pro plate reader.

Other assays are known in the art, see for example, Lempp et al., Nature Communications, 2019, 10:2265, https://doi.org/10.1038/s41467-019-10211-2, Grosser et al., Frontiers in Molecular Biosciences, 2021, 8: doi: 10.3389/fmolb.2021.689757.

Table 2 provides assay data for exemplified compounds of the invention grouped in the following ranges: A indicates EC₅₀<100 nM; B indicates EC₅₀ of ≥100 to <1,000 nM; C indicates EC₅₀ of ≥1,000 to <5,000 nM.

TABLE 2
Assay data for exemplified compounds of the invention.

| Example | HBeAg EC₅₀ (μM) |
|---|---|
| 1 | B |
| 2 | A |
| 2a | A |
| 2b | B |
| 3 | A |
| 4 | C |
| 5 | B |
| 8 | A |
| 9 | A |
| 10 | A |
| 11 | A |
| 12 | B |
| 13 | B |
| 14 | B |
| 15 | B |
| 16 | A |
| 17 | A |
| 18 | A |
| 19 | B |
| 20 | A |
| 21 | B |
| 22 | C |
| 23 | B |
| 24 | B |
| 25 | A |
| 26 | B |
| 27 | A |
| 28 | A |
| 29 | B |
| 30 | A |
| 31 | B |
| 32 | A |
| 33 | A |
| 34 | A |
| 35 | B |
| 36 | A |
| 37 | A |
| 38 | B |
| 39 | C |
| 40 | A |
| 41 | C |
| 42 | B |
| 43 | A |
| 44 | B |
| 45 | C |
| 46 | C |
| 47 | C |
| 48 | C |
| 49 | B |
| 50 | B |
| 51 | C |
| 52 | A |
| 53 | B |
| 54 | C |
| 55 | C |
| 56 | A |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A compound of Formula II 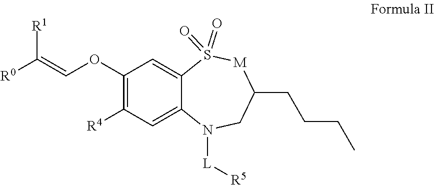
or a pharmaceutically acceptable salt thereof, wherein:
L is C₁₋₄alkylene or C(O);
M is NR^{x} or CR²R³;
R^{x} is hydrogen or C₁₋₄alkyl;
R⁰ is —C(O)OH, —C(O)OC₁₋₆alkyl, or —S(O)₂OH;
R¹ is selected from the group consisting of hydrogen, halo, C₁₋₄alkyl and haloC₁₋₄alkyl;
R² and R³ are independently selected from the group consisting of hydrogen, halo and methyl;
R⁴ is independently selected from the group consisting of hydrogen, halo, cyano, C₁₋₄alkyl, haloC₁₋₄alkyl, C₁₋₄alkoxy and C₁₋₄alkylthio;
R⁵ is an C₁₋₆alkyl, C₂₋₆alkenyl, C₂₋₆alkynyl, haloC₁₋₆alkyl, C₃₋₇monocycloalkyl, -L-C₃₋₇monocycloalkyl, mono₃₋₇heterocycloalkyl, -L-mono₃₋₇heterocycloalkyl, -L-phenyl, monocyclo₅₋₆heteroaryl or -L-monocyclo₅-6heteroaryl group, wherein the C₃₋₇monocycloalkyl, -L-C₃₋₇monocycloalkyl, mono₃₋₇heterocycloalkyl, -L-mono₃₋₇heterocycloalkyl, -L-phenyl, monocyclo₅₋₆heteroaryl or -L-monocyclo₅s-heteroaryl group is optionally substituted with 1-3 substituents independently selected from the group consisting of halo, OH, CN, C₁₋₄alkyl, haloC₁₋₄alkyl, C₁₋₄alkoxy and C₁₋₄alkylthio.

2. The compound of claim 1, or a pharmaceutically acceptable salt thereof, wherein M is NR^{x}.

3. The compound of claim 1 or 2, or a pharmaceutically acceptable salt thereof, wherein M is NH or NCH₃.

4. The compound according to any one of claims 1-3, or a pharmaceutically acceptable salt thereof, wherein M is CH₂, CF or CF₂.

5. The compound according to any one of claims 1-4, or a pharmaceutically acceptable salt thereof, wherein R⁰ is —C(O)OH or —C(O)OC₁₋₄alkyl.

6. The compound of claim 5, or a pharmaceutically acceptable salt thereof, wherein R⁰ is —C(O)OH.

7. The compound according to any one of claims 1-6, or a pharmaceutically acceptable salt thereof, wherein R¹ is hydrogen or F.

8. The compound of claim 7, or a pharmaceutically acceptable salt thereof, wherein R¹ is F.

9. The compound according to any one of claims 1-8, or a pharmaceutically acceptable salt thereof, wherein R⁴ is methylthio.

10. The compound according to any one of claims 1-9, or a pharmaceutically acceptable salt thereof, wherein R⁵ is C₁₋₆alkyl, C₂₋₆alkenyl, C₂₋₆alkynyl or haloC₁₋₆alkyl.

11. The compound according to any one of claims 1-9, or a pharmaceutically acceptable salt thereof, wherein R⁵ is R⁵ is C₃₋₇monocycloalkyl, -L-C₃₋₇monocycloalkyl, mono₃₋₇heterocycloalkyl or -L-mono₃₋₇heterocycloalkyl group, wherein the C₃₋₇monocycloalkyl, -L-C₃₋₇monocycloalkyl, mono₃₋₇heterocycloalkyl or -L-mono₃₋₇heterocycloalkyl group is optionally substituted with 1-3 substituents independently selected from the group consisting of halo, OH, CN, C₁₋₄alkyl, haloC₁₋₄alkyl, C₁₋₄alkoxy and C₁₋₄alkylthio.

12. The compound of claim 11, or a pharmaceutically acceptable salt thereof, wherein R⁵ is C₃₋₇monocycloalkyl or mono₃₋₇heterocycloalkyl, wherein the C₃₋₇monocycloalkyl or mono₃₋₇heterocycloalkyl is optionally substituted with 1-3 substituents independently selected from the group consisting of halo, OH, CN, C₁₋₄alkyl, haloC₁₋₄alkyl, C₁₋₄alkoxy and C₁₋₄alkylthio.

13. The compound of claim 12, or a pharmaceutically acceptable salt thereof, wherein R⁵ is C₃₋₇monocycloalkyl group optionally substituted with 1-3 substituents independently selected from the group consisting of halo, OH, CN, C₁₋₄alkyl, haloC₁₋₄alkyl, C₁₋₄alkoxy and C₁₋₄alkylthio.

14. The compound of claim 12, or a pharmaceutically acceptable salt thereof, wherein R⁵ is a mono₃₋₇heterocycloalkyl group optionally substituted with 1-3 substituents independently selected from the group consisting of halo, OH, CN, C₁₋₄alkyl, haloC₁₋₄alkyl, C₁₋₄alkoxy and C₁₋₄alkylthio.

15. The compound of claim 14, or a pharmaceutically acceptable salt thereof, wherein R⁵ is selected from the group consisting of 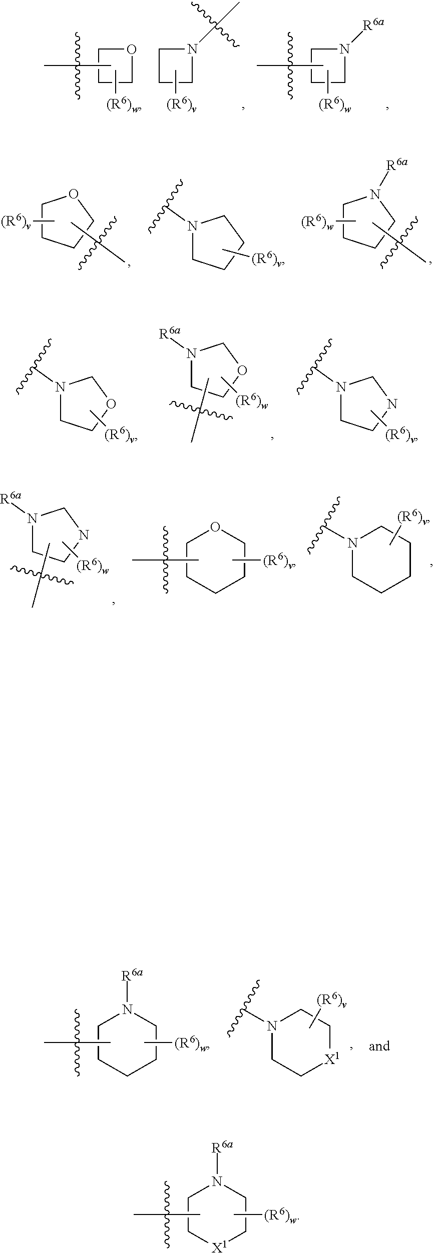
wherein:
R⁶ is independently selected for each occurrence from the group consisting of halo, OH, CN, C₁₋₄alkyl, haloC₁₋₄alkyl, C₁₋₄alkoxy and C₁₋₄alkylthio;
R^{6a} is hydrogen or C₁₋₆alkyl;
X¹ is O, S or NR⁶;
v is 0, 1, 2 or 3; and
w is 0, 1 or 2.

16. The compound according to any one of claims 1-9, or a pharmaceutically acceptable salt thereof, wherein R⁵ is a -L-phenyl group optionally substituted with 1-3 substituents independently selected from the group consisting of halo, OH, CN, C₁₋₄alkyl, haloC₁₋₄alkyl, C₁₋₄alkoxy and C₁₋₄alkylthio.

17. The compound according to any one of claims 1-9, or a pharmaceutically acceptable salt thereof, wherein R⁵ is monocyclo₅₋₆heteroaryl or -L-monocyclo₅₋₆heteroaryl group, wherein the monocyclo₅₋₆heteroaryl or -L-monocyclo₅₋₆heteroaryl group is optionally substituted with 1-3 substituents independently selected from the group consisting of halo, OH, CN, C₁₋₄alkyl, haloC₁₋₄alkyl, C₁₋₄alkoxy and C₁₋₄alkylthio.

18. The compound of claim 17, or a pharmaceutically acceptable salt thereof, wherein R⁵ is 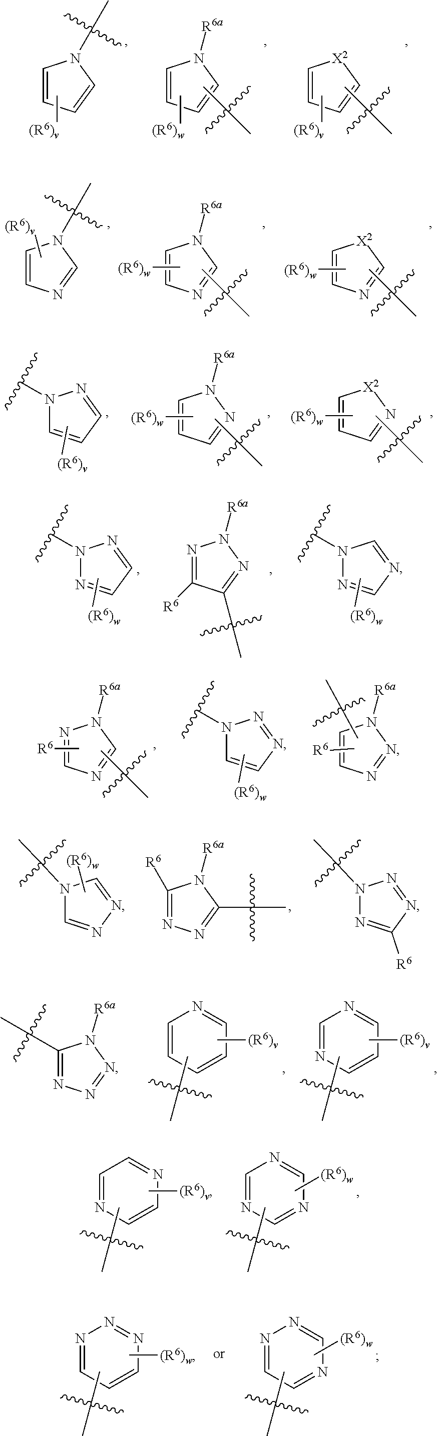
R⁶ is independently selected for each occurrence from the group consisting of halo, OH, CN, C₁₋₄alkyl, haloC₁₋₄alkyl, C₁₋₄alkoxy and C₁₋₄alkylthio;
R^{6a} is hydrogen or C₁₋₆alkyl;
X² is O or S;
v is 0, 1, 2 or 3; and
w is 0, 1 or 2.

19. A compound, or a pharmaceutically acceptable salt thereof, selected form the group consisting of

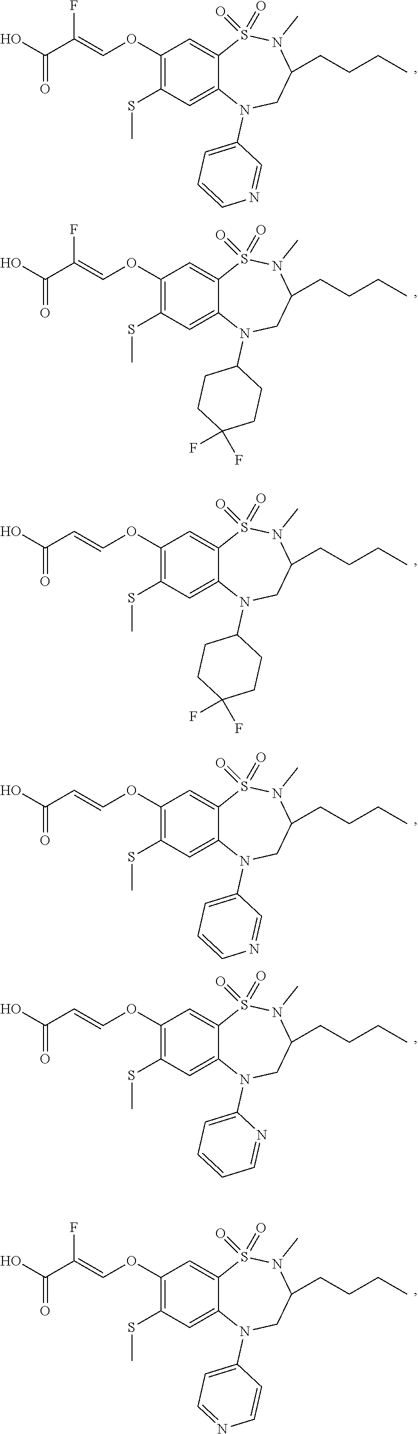

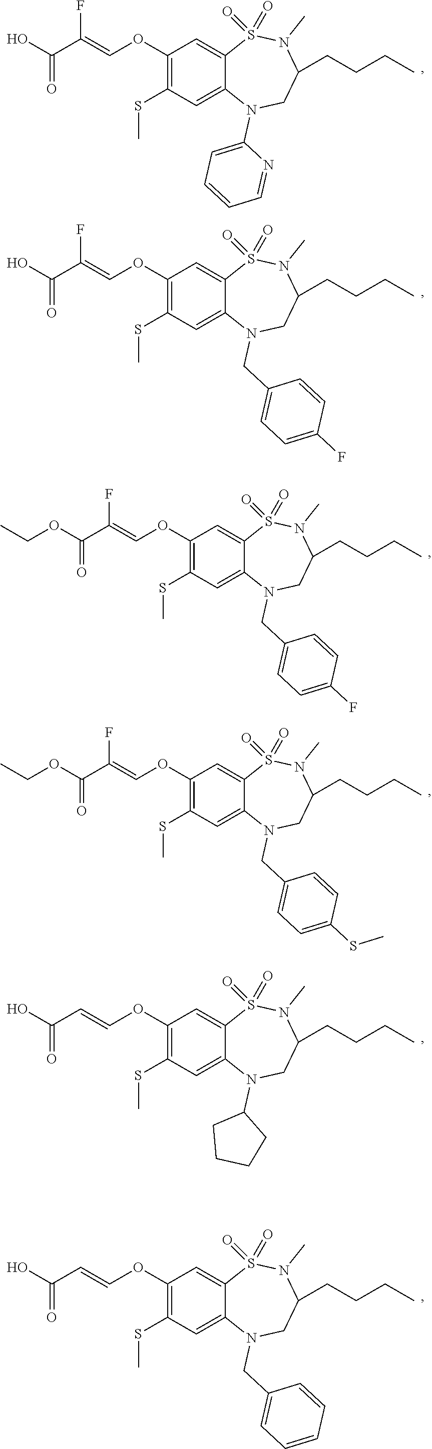

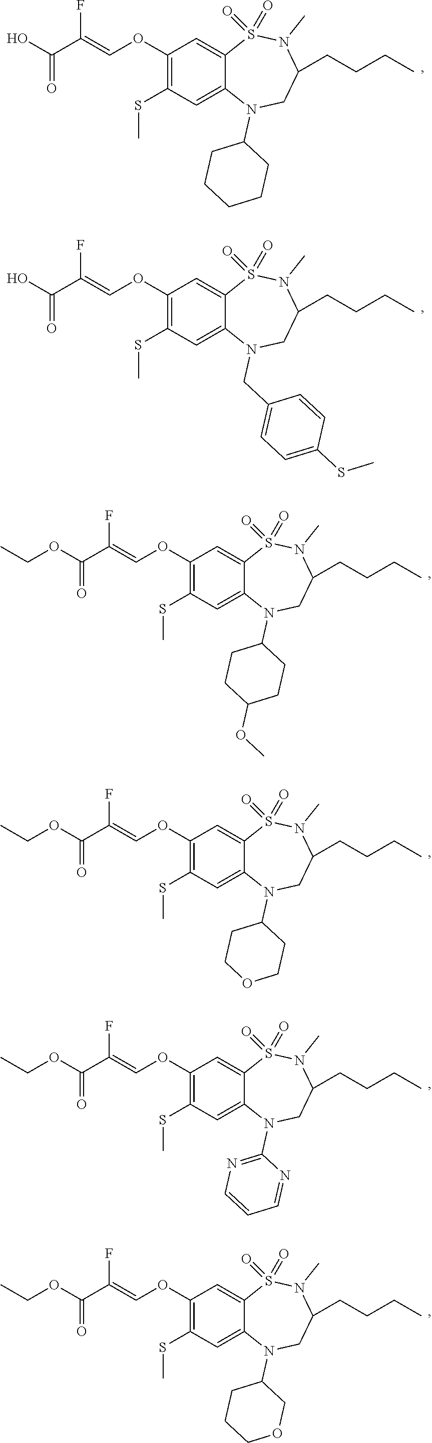

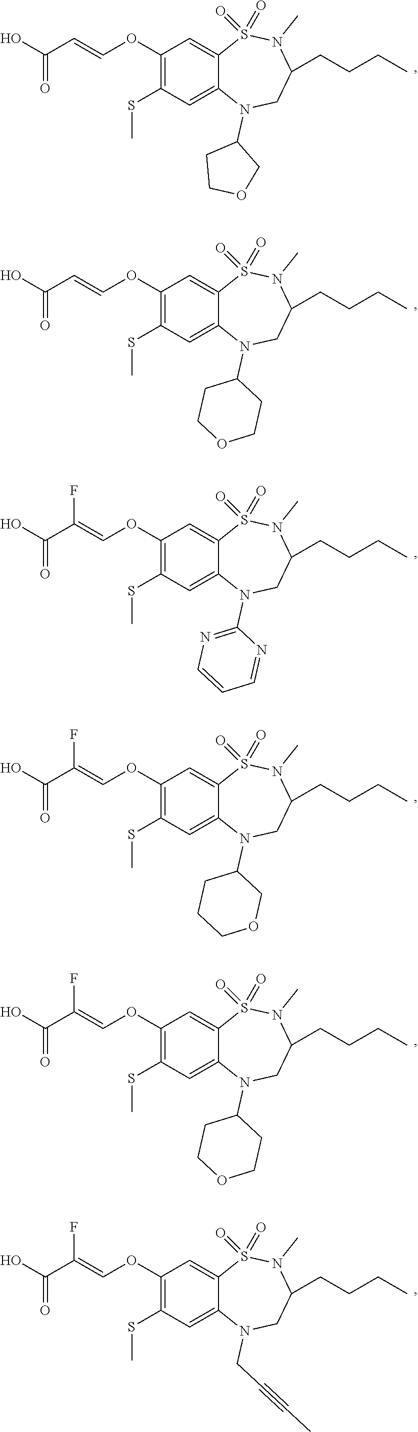 [SMILES: CCCCC1CN(C2CCOC2)c2cc(SC)c(O/C=C/C(=O)O)cc2S(=O)(=O)N1C ; CCCCC1CN(C2CCOCC2)c2cc(SC)c(O/C=C/C(=O)O)cc2S(=O)(=O)N1C ; CCCCC1CN(c2ncccn2)c2cc(SC)c(O/C=C(\F)C(=O)O)cc2S(=O)(=O)N1C ; CCCCC1CN(C2CCCOC2)c2cc(SC)c(O/C=C(\F)C(=O)O)cc2S(=O)(=O)N1C ; CCCCC1CN(C2CCOCC2)c2cc(SC)c(O/C=C(\F)C(=O)O)cc2S(=O)(=O)N1C ; CC#CCN1CC(CCCC)N(C)S(=O)(=O)c2cc(O/C=C(\F)C(=O)O)c(SC)cc21]

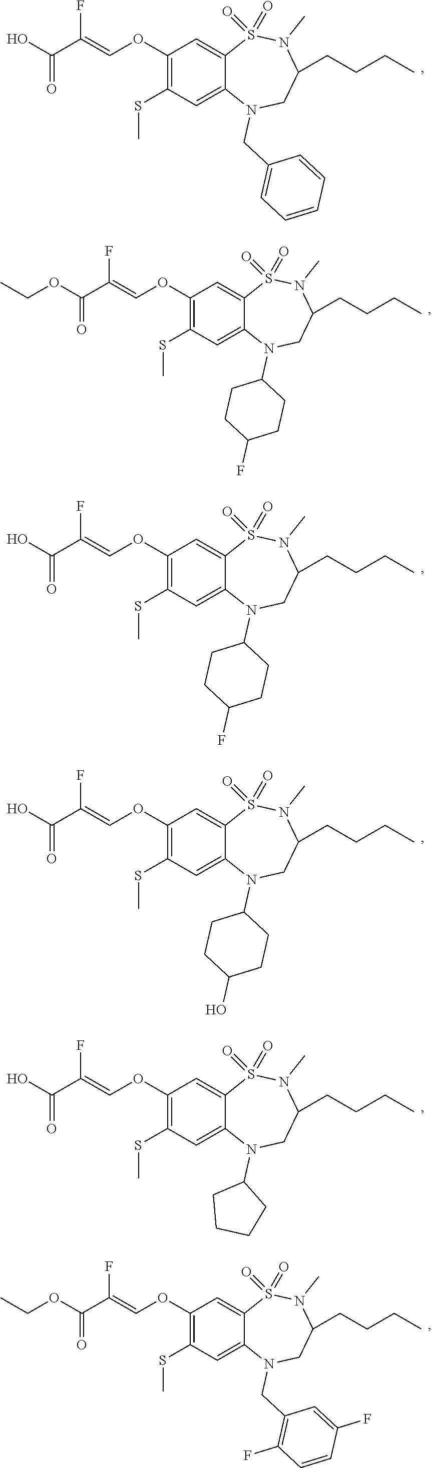

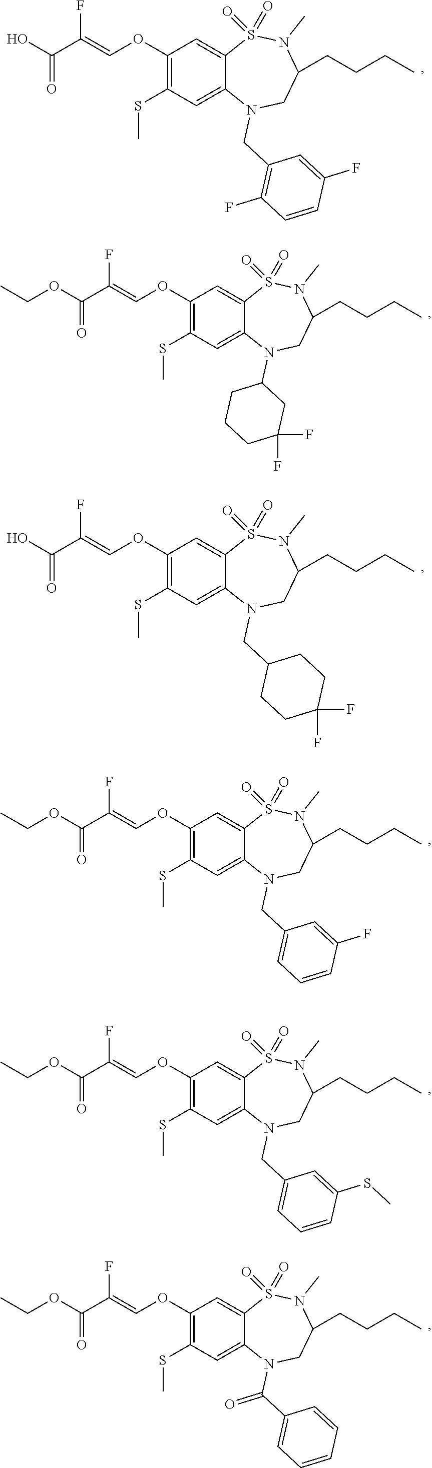

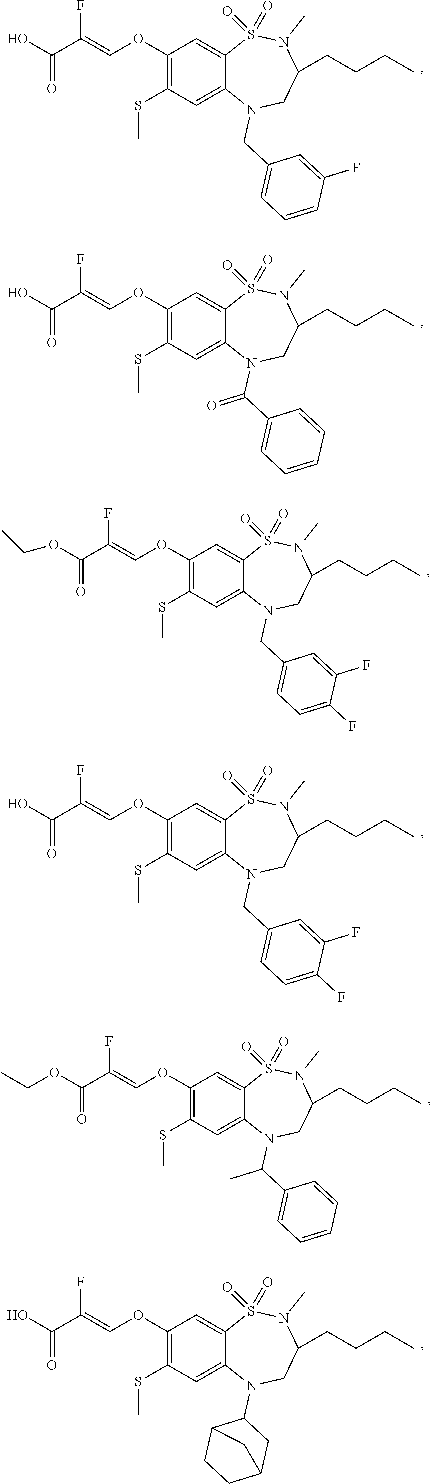

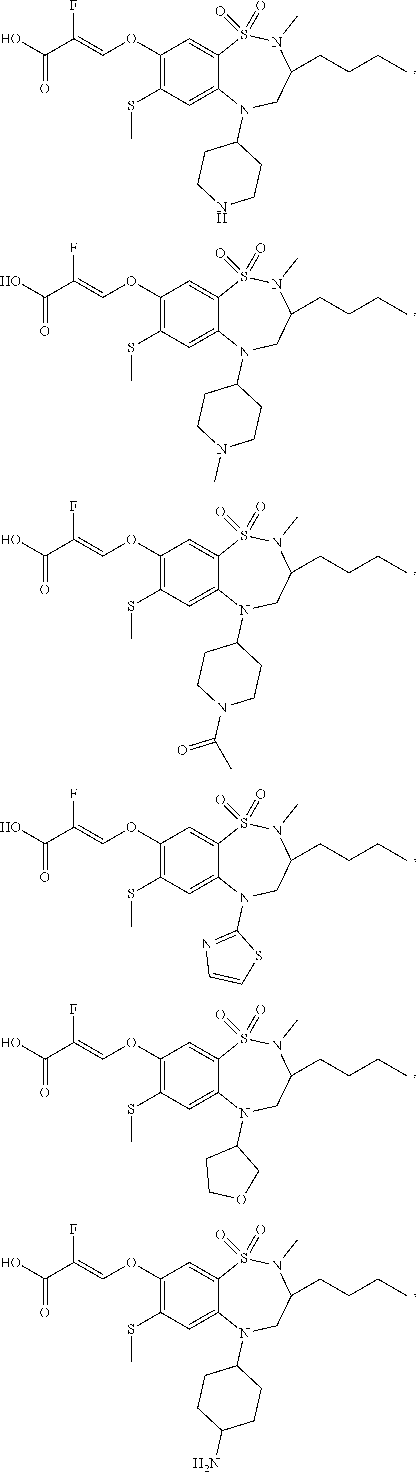

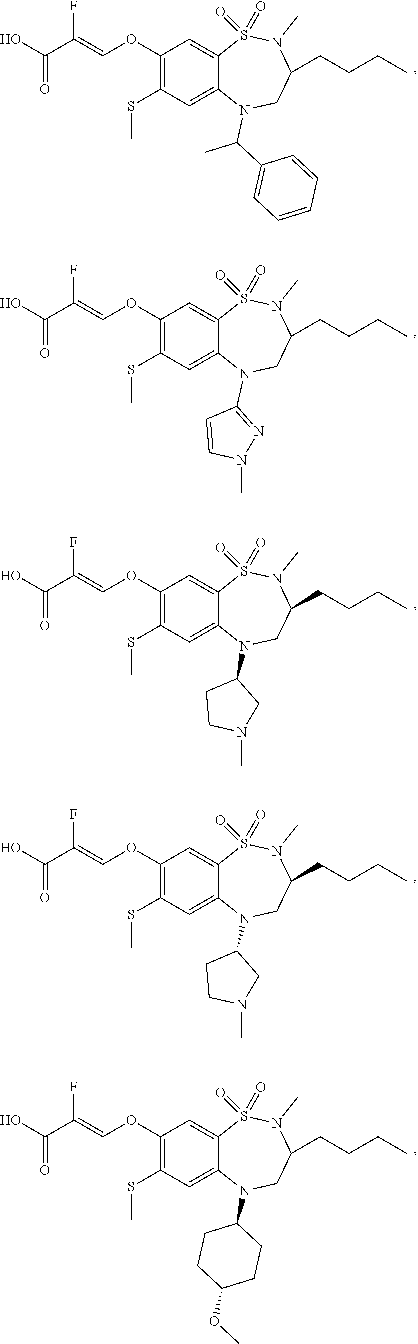 [SMILES: CCCCC1CN(C(C)c2ccccc2)c2cc(SC)c(O/C=C(\F)C(=O)O)cc2S(=O)(=O)N1C ; CCCCC1CN(c2ccn(C)n2)c2cc(SC)c(O/C=C(\F)C(=O)O)cc2S(=O)(=O)N1C ; CCCC[C@H]1CN([C@@H]2CCN(C)C2)c2cc(SC)c(O/C=C(\F)C(=O)O)cc2S(=O)(=O)N1C ; CCCC[C@H]1CN([C@H]2CCN(C)C2)c2cc(SC)c(O/C=C(\F)C(=O)O)cc2S(=O)(=O)N1C ; CCCCC1CN([C@H]2CC[C@H](OC)CC2)c2cc(SC)c(O/C=C(\F)C(=O)O)cc2S(=O)(=O)N1C]

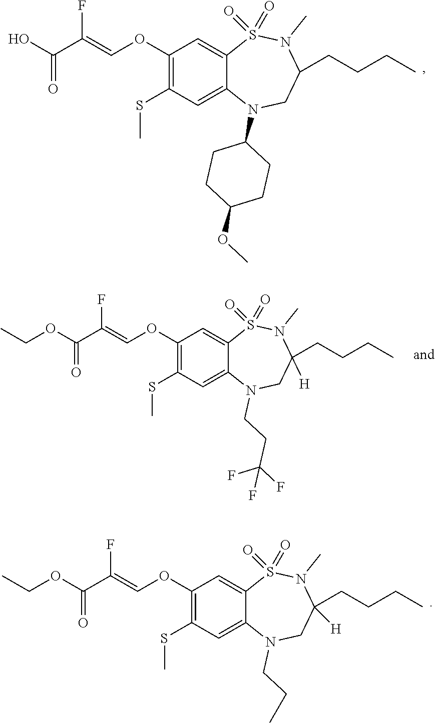

20. A pharmaceutical composition comprising a compound according to any one of claims 1-19, or a pharmaceutically acceptable salt thereof, and a pharmaceutically acceptable excipient.

21. A method of treating Hepatitis B (HBV) infection in a subject in need thereof, the method comprising: administering to the subject a therapeutically effective amount of a compound according to any one of claims 1-19, or a pharmaceutically acceptable salt thereof.

22. A method of treating Hepatitis B (HBV) infection in a subject in need thereof, the method comprising: administering to the subject a therapeutically effective amount of a pharmaceutical composition of claim 20.

23. A method of treating Hepatitis D (HDV) infection in a subject in need thereof, the method comprising: administering to the subject a therapeutically effective amount of a compound according to any one of claims 1-19, or a pharmaceutically acceptable salt thereof.

24. A method of treating Hepatitis D (HDV) infection in a subject in need thereof, the method comprising: administering to the subject a therapeutically effective amount of a pharmaceutical composition of claim 20.
